(19) United States
(12) Patent Application Publication
(10) Pub. No.: US 2023/0189773 A1
(43) Pub. Date: Jun. 22, 2023

(54) ENGINEERED CELLS, ANIMAL MODELS, AND USES THEREOF FOR MODELING LOW GRADE GLIOMA (LGG)

(71) Applicants: David Gutmann, St. Louis, MO (US); Corina Anastasaki, St. Louis, MO (US)

(72) Inventors: David Gutmann, St. Louis, MO (US); Corina Anastasaki, St. Louis, MO (US)

(73) Assignee: Washington University, St. Louis, MO (US)

(21) Appl. No.: 18/069,358

(22) Filed: Dec. 21, 2022

Related U.S. Application Data
(60) Provisional application No. 63/292,012, filed on Dec. 21, 2021.

(51) Int. Cl. A01K 67/027; C12N 5/079; G01N 33/50

(52) U.S. Cl. CPC A01K 67/0278; C12N 5/0622; G01N 33/5088; G01N 33/5011; C12N 2506/45; C12N 2500/38; C12N 2501/235; C12N 2501/33; C12N 2501/105; C12N 2501/115; A01K 2207/15; A01K 2207/12; A01K 2217/056; A01K 2227/105; A01K 2267/0331

(57) ABSTRACT

Among the various aspects of the present disclosure is the provision of engineered cells, animal models, and uses thereof for modeling low grade glioma (LGG). An aspect of the present disclosure provides for a population of cells engineered to silence, downregulate, knock out, or reduce or knock down Cxcl10 expression. Another aspect of the present disclosure provides for an animal engineered to be deficient in Cxcl10, downregulate or reduce expression of Cxcl10, knock out Cxcl10, or knock down Cxcl10 (e.g., Cxcl10^{-/-} mice). Yet another aspect of the present disclosure provides for a method of growing tumor cell lines or patient-derived xenografts for LGG tumors in an animal (e.g., mouse, rat) including providing a mouse or rat harboring somatic homozygous deletion in the Rag1 or Cxcl10 gene, and implanting an amount of the cells in mice sufficient to grow a tumor.

### CROSS-REFERENCE TO RELATED APPLICATIONS

This application claims priority from U.S. Provisional Application Ser. No. 63/292,012 filed on 21 Dec. 2021, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD

The present disclosure generally relates to engineered cells for use in animal models.

### SUMMARY

Among the various aspects of the present disclosure is the provision of engineered cells, animal models, and uses thereof for use in modeling low grade glioma (LGG). An aspect of the present disclosure provides for the development of human brain tumor models in which cells (e.g., patient-derived primary human PA cell lines, hiPSCs) to be grown in rodents engineered to silence, downregulate, knock out, or reduce or knock down Cxcl10 expression (e.g., Cxcl10^{-/-}). Another aspect of the present disclosure provides for an animal (e.g., mouse, rat) model comprising: an animal engineered to be deficient in Cxcl10, downregulate or reduce expression of Cxcl10, knock out Cxcl10, or knock down Cxcl10 (e.g., Cxcl10^{-/-} mice). Yet another aspect of the present disclosure provides for a method of growing patient-derived xenografts for LGG tumors in an animal (e.g., mouse, rat) comprising: providing a mouse or rat harboring somatic homozygous deletion in the Rag1 or Cxcl10 gene; and implanting an amount of patient-derived LGG cells (e.g., patient tumor-derived cells) in mice sufficient to grow a tumor. Yet another aspect of the present disclosure provides for a method of growing cell-derived xenografts for LGG-like tumors in mice/rats comprising: providing a mouse or rat harboring somatic homozygous deletion in the Rag1 or Cxcl10 gene; and implanting an amount of human cell-derived LGG cells (e.g., human stem cells, tumor-derived cells) in mice sufficient to grow a tumor. In some embodiments, the population of cells are selected from human induced pluripotent stem cell (hiPSC); human pediatric LGG cell lines; human neuroglial lineage cells; are not astrocytoma cells; primary human PA cell lines from a patient with an NF1-PA (JHH-NF-PA; NF1 loss) or with a sporadic PA (Res186; KIAA1549:BRAF fusion); NF1-null and KIAA1549:BRAF human induced pluripotent stem cell (hiPSC)-derived neural progenitor cells (iNPCs) (i.e., iNPCs with NF1 loss or KIAA1549:BRAF expression); iPSC-derived glial restricted progenitors (iGRPs) (e.g., CD133+, SOX2+, O4+, GFAP+ and S100β+ cells); oligodendrocyte progenitors (iOPCs) (e.g., O4+, MBP+, GFAP+ and NG2neg cells); or JHH-NF-PA, Res186, WUPA1, WUPA2 and WUPA3 primary cell lines derived from patient-resected pilocytic astrocytoma tumors. In some embodiments, the population of cells are generated by hiPSC engineering. In some embodiments, the cell-derived xenografts for LGG-like tumors are selected from: human iPSCs harboring mutations in the NF1 gene (e.g., deletions, point mutations such as c.2041C>T and c.6513T>A); or human iPSCs expressing the KIAA1549:BRAF fusion gene. In some embodiments, the animal model comprises a population of human cells in the brain of the animal. In some embodiments, the population of human cells is patient-derived or iGRPs, iOPCs, or iNPCs with NF1 loss or KIAA1549:BRAF expression. In some embodiments, the model is a patient-derived xenograft (PDX) model. In some embodiments, the xenograft is a pLGG xenograft and develops glioma-like lesions in the animal brain. Yet another aspect of the present disclosure provides for a method of making cells comprising: obtaining human iPS cells; and introducing NF1 gene mutations into these hiPSC cells. In some embodiments, the mutations in NF1 are c.2041C>T; c.6513T>A. Yet another aspect of the present disclosure provides for a method of making an animal (e.g., mouse, rat) model of low grade glioma comprising: (i) obtaining a Cxcl10-deficient animal (e.g., mouse, rat) (genetically engineered to have reduced or eliminated Cxcl10); (ii) intracranially injecting a cell population (e.g., 1×10⁴, 1×10⁴, 1×10⁵ or 5×10⁵ cells resuspended in 2 μL ice-cold PBS were injected 0.7 mm to the right of the midline into either the midbrain (0.5 mm posterior to Lambda; 2 mm deep), or the cerebellum (2 mm posterior to Lambda; 1 mm deep)) into the brain of mice, optionally neonatal mice. Yet another aspect of the present disclosure provides for a method of identifying putative cells of origin for tumors comprising injecting cells suspected of being a cell of origin into an animal (e.g., mouse, rat) brain and determining the tumor type. Yet another aspect of the present disclosure provides for a method of determining putative cells of origin for histologically-distinct tumors, as well as histologically-similar tumors arising in different locations origin using the hiPSC-LGG explant system comprising: testing different cells in the model and determining if they survive. Yet another aspect of the present disclosure provides for a method of making an animal model capable of growing tumors in the brain comprising injecting pups with Cxcl10^{-/-} strain. In some embodiments, the method further comprises (i) CRISPR/Cas9-engineering a NF1 patient homozygous and heterozygous germline NF1 gene (Transcript ID NM_000267) mutations (c.2041C>T; c.6513T>A) into a single commercially available male control human iPSC line (e.g., BJFF.6) and incubated/cultured; or (ii) differentiating hiPSCS into neural progenitor cells (iNPCs) comprising hiPSCs were transferred to poly-L-ornithine/Laminin-coated tissue culture flasks and incubated in NPC basic media supplemented with human LIF, CHIR99021, SB431542, Dorsomorphin and Compound E; next, incubating in NPC basic medium supplemented with human LIF, CHIR99021, SB431542, and Compound E; next, iNPCs were incubated and maintained in NPC basic medium supplemented with human LIF, CHIR99021 and SB431542; (iii) differentiating iNPCs into glial restricted progenitor (iGRP) comprising dissociating iNPCs with Accutase, and floating cells transferred to low-attachment culture flasks to allow for gliosphere formation; incubating iGRPs in the following medium comprising Basal GRP medium supplemented with NT-3, forskolin, 3,3′,5-triiodo-L-thyronine, ascorbic acid and insulin; or (iv) differentiating iPSCs into oligodendrocyte progenitor cell (OPC) comprising generating embryoid bodies (EBs) comprising seeding iPSCs at the bottoms of ultra-low cell attachment vessel, and incubating in NIM; transferring EBs onto poly-L-ornithine/ Laminin-coated vessel and incubated in NIM supplemented with bFGF and heparin; then incubating in NIM supplemented with retinoic acid; incubating in NIM supplemented with RA, Purmorphamine and 1×B-27; and then incubating in NIM supplemented with bFGF, Pur and 1×B-27; maturing into OPCs, comprising transferring the spheres to low-attachment culture vessel and incubated in glial induction medium supplemented with PDGF-AA, IGF-1 and NT3. In some embodiments, the low grade glioma model is a humanized NF1-associated and sporadic KIAA1549:BRAF-driven pediatric low-grade glioma (pLGG)-like model. In some embodiments, the methods, cell population, or animal model is for use in screening platform for therapeutic drug testing. In some embodiments, the cells are from a patient having or, the cells, when implanted, model: slow-growing neoplasms located in the cerebellum, brainstem, or optic pathway; low-grade glioma (e.g., pediatric LGG (pLGG) such as grade 1 pilocytic astrocytoma (PA)); NF1-associated optic pathway glioma (NF1-OPG); or BRAF-driven sporadic pLGG. In some embodiments, the in vivo patient-derived pLGG xenograft model is used for patient-specific care and iHSCs are for use in off-the-shelf applications. In some embodiments, the cells do not have genetic mutations such as TP53/CDKNIA and CDKN2A/RB1 alterations (which are uncharacteristic of childhood gliomas, especially PAs). In some embodiments, the cells are NF1-null and KIAA1549:BRAF human induced pluripotent stem cell (hiPSC)-derived neural progenitor cells (iNPCs). In some embodiments, the animal models NF1 loss of heterozygosity in pLGG and the cells are engineered to have c.2041C>T^{-/-} or c.6513T>A^{-/-} NFL1 mutation. In some embodiments, neuronal and glial lineage cells are generated from NF1-null and control hiPSCs differentiated into multipotent human neural stem cells (iNPCs). In some embodiments, the low grade glioma model is a sporadic pLGG resulting from genomic rearrangements involving the BRAF kinase gene and the cells are KIAA1549:BRAF-expressing iNPCs. In some embodiments, the low grade glioma model is detectable by magnetic resonance imaging (MRI). In some embodiments, MRI is used to monitor tumor progression in response to treatment or treatment efficacy. In some embodiments, pathology or histology is used to monitor tumor progression in response to treatment or treatment efficacy. In some embodiments, the cells or the animal model generates tumors having one or more of the following characteristics: the tumors are composed of human cells (e.g., Ku80⁺ cells), are hypercellular, have tumor cells located in parenchymal, have exophytic components either anterior or lateral to the midbrain/brainstem tissue (e.g., in NF1-null iNPC tumors) or anterior to the cerebellum (e.g., in KIAA1549:BRAF-iNPC tumors), and are well-circumscribed. In some embodiments, the cells or the animal model generates tumors having the following characteristics: the tumors contain GFAP- and OLIG2-immunopositive cells (e.g., as seen in pediatric LGGs). In some embodiments, the cells or the animal model generates tumors having the following characteristics: the iNPC-lesions contained both glioma-like areas, as determined by H&E, GFAP and OLIG2 (glial) immunopositivity, and embryonal-like hypercellular neuronal (synaptophysin+) areas, optionally containing neuroepithelial rosettes. In some embodiments, the cells or the animal model generates tumors having the following characteristics: Ku80⁺ cells in these lesions express CD133 and ABCG1, markers of glioma stem cells, and optionally immunonegative for SOX10 and p16 expression. In some embodiments, iNPCs generate both NF1-associated and sporadic LGG-like lesions in vivo. In some embodiments, hiPSC-derived glial restricted progenitors (iGRPs) (e.g., CD133+, SOX2+, O4+, GFAP+ and S100β+ cells) and oligodendrocyte progenitors (iOPCs) (e.g., O4+, MBP+, GFAP+ and NG2neg cells), form LGG-like lesions in Rag1^{-/-} mice. In some embodiments, tumors exhibited low proliferative indexes. In some embodiments, cells form LGG-like lesions in the cerebellum, midbrain/brainstem, or the hippocampus. In some embodiments, the tumors generated model sporadic and NF1-associated pLGGs. In some embodiments, the animal is an immunodefective animal (e.g., mouse, rat) strain (e.g., Cxcl10), permits LGG-like lesion formation, and is not a wild type animal. In some embodiments, the immunodefective animal (e.g., immunodeficient animal, mouse, rat) strain is Cxcl10^{-/-}. In some embodiments, the immunodefective animal (e.g., mouse, rat) strain is Rag1^{-/-} mice having Chil3, Cd59, and Cxcl10 downregulated transcripts. In some embodiments, the immunodefective animal (e.g., mouse, rat) strain is NOD/SCID, CD4-deficient, or CD4/CD8-deficient animal. In some embodiments, the immunodefective animal (e.g., mouse, rat) strain is not CD8-deficient mice or strains deficient in the expression of microglia or T cell chemokine receptors (Cx3cr1, Ccr2). In some embodiments, the animal model is a PDX model and the animal (e.g., mouse, rat) strain is Cxc110⁻deficient

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1(A-F) shows an exemplary embodiment of characterization of hiPSCs and iNPCs in accordance with the present disclosure. FIG. 1A contains images showing NF1-null (2041C>T^{-/-}, 6513T>A^{-/-}) and control hiPSCs are immunopositive for OCT4A, Nanog, SOX2, TRA-1-60, TRA-1-81, and SSEA4 (pluripotency markers) expression (top panel). 2041C>T^{-/-}, 6513T>A^{-/-} and control iNPCs express SOX2, BLBP and Nestin, and can be differentiated into TUJ1⁺ neurons and S100β⁺ glial cells (bottom panel). Scale bars, 25 μm. FIG. 1B is a bar graph showing RAS activity is increased by 2.2- and 1.8-fold in heterozygous NF1-mutant iNPCs (2041C>T^{+/-} and 6513T>A^{+/-}), respectively, and by 9.2- and 13.9-fold in NF1-null iNPCs (2041C>T^{-/-} and 6513T>A^{-/-}), respectively, compared to controls. n=3. FIG. 1C is a bar graph showing cAMP levels are equivalently reduced in heterozygous NF1-mutant (2041C>T^{+/-}: 49%; 6513T>A^{+/-}: 61%) and NF1-null (2041C>T^{-/-}: 51%; 6513T>A^{-/-}: 57%) iNPCs relative to controls. n=3. FIG. 1D is a bar graph showing BrdU incorporation (proliferation; n=8) is increased 3.3- and 3.5-fold in heterozygous NF1-mutant iNPCs (2041C>T^{+/-} and 6513T>A^{+/-}) iNPCs, 7.9- and 8.2-fold in NF1-null iNPCs (2041C>T^{-/-} and 6513T>A^{-/-}) and 6.5-fold in KIAA1549:BRAF iNPCs, respectively, compared to controls. FIG. 1E is a bar graph showing a 1.8- and 2.0-fold increase in direct cell counting (n=4) in heterozygous NF1-mutant iNPCs (2041C>T^{+/-} and 6513T>A^{+/-}), a 4.2- and 4.6-fold increase in NF1-null iNPCs (2041C>T^{-/-} and 6513T>A^{-/-}), and 4-fold in KIAA1549:BRAF iNPCs relative to controls. FIG. 1F contains western immunoblots demonstrating increased phospho-ERK1/2 normalized to total ERK1/2 in KIAA1549:BRAF-iNPCs and isogenic controls (CTLs). Two independently generated clones for each of the iNPC lines are included. α-tubulin was used as a protein loading control. All data are represented as means ±SEM; one-way ANOVA with Bonferroni post-test correction. Individual p values are indicated within each graph.

FIG. 2 contains a table and images depicting analysis of iNPC-injected Rag1^{-/-} mice in accordance with the present disclosure. The table shows a summary of the percentages of Rag1^{-/-} mice harboring LGGs one month after injection of 5×10⁵, 1×10⁵, 5×10⁴ and 1×10⁴ 2041C>T^{-/-} or 6513T>A^{-/-} NF1-null iNPCs. The total number of mice injected is shown in the parentheses. The images are representative images of Ku80⁺ and Ki67⁺ cells in Rag1^{-/-} mouse brainstems one month after injection of 1×10⁵ 2041C>T^{-/-} and 6513T>A^{-/-} NF1-null iNPCs. White arrowheads indicate Ku80⁺ (human) cells.

FIG. 3(A-D) is an exemplary embodiment showing Rag1^{-/-} mice develop NF1-null and KIAA1549:BRAF-expressing low-grade gliomas (LGGs) in accordance with the present disclosure. FIG. 3A contains a schematic and images showing injection of NF1-null iNPCs (2041C>T^{-/-}, 6513T>A^{-/-}) into the brainstems or KIAA1549:BRAF-expressing iNPCs into the cerebella of Rag1^{-/-} mice result in the formation of LGGs detectable by MRI (denoted by red dotted lines). FIG. 3B is a table showing a summary of injected Rag1^{-/-} mice harboring iNPC LGGs. FIG. 3C contains images showing LGGs are hypercellular (H&E staining) and immunopositive for Ku80 (human-specific antibody), Ki67, glial (GFAP, OLIG2) and neuronal (synaptophysin) marker expression, by 1 month post-injection (mpi). The tumors had both glial only (top panels) and mixed embryonal/glial (lower panels) histological characteristics. Scale bars: left, Ku80/H&E panels, 1 mm; other panels, 100 μm. FIG. 3D contains images showing LGGs contain CD133⁺, ABCG1⁺, PDPN⁺, BLBP, and GFAP⁺ cells (white arrowheads). No Ku80⁺ LGG tumor cells had SOX10 or p16 expression. Scale bar, 100 μm.

FIG. 4(A-D) shows an exemplary embodiment of analysis of iNPC-injected Rag1^{-/-} mice in accordance with the present disclosure. FIG. 4A contains images showing iNPC LGGs are negative for non-specific pluripotency markers OCT4 and Nanog, negative for endoderm and mesoderm markers SMA and AFP, and immunopositive for neural progenitor pluripotency marker Nestin. FIG. 4B is an image showing increased ERK1/2 phosphorylation (activation; pERK1/2, red) observed in KIAA1549:BRAF-iNPC LGG tumor cells (Ku80⁺ cells, green) relative to the surrounding normal brain tissue (Ku80-negative). Dashed white lines indicate the lesion area. FIG. 4C contains a table and images showing Rag1^{-/-} mice do not develop LGGs 1 month post-injection (m.p.i.) of 5×10⁵ CTL, 2041C>T^{+/-} and 6513T>A^{+/-} (heterozygous NF1-mutant) iNPCs. The lower panel shows representative images of Ku80⁺, Ki67⁺ and GFAP^{neg} CTL iNPCs at the identified injection site. White arrowheads indicate Ku80⁺ iNPCs. FIG. 4D contains bar graphs showing PDPN and FABP7 are differentially expressed in NF1-associated and sporadic (Sp-PAs) pilocytic astrocytomas (PAs) relative to non-neoplastic brain tissue (CTL). R.E., relative expression. Scale bar is 100 μm for FIG. 4A and 50 μm for FIG. 4B-FIG. 4C.

FIG. 5(A-D) is an exemplary embodiment showing Rag1^{-/-} mice develop persistent LGGs in accordance with the present disclosure. FIG. 5A is a table showing percentage of Rag1^{-/-} mice harboring LGGs at 1, 3 and 6 mpi. The total number of injected mice is denoted in the parentheses. G, glial only; G/E, mixed glial/embryonal. FIG. 5B contains images showing representative H&E staining of NF1-null and KIAA1549:BRAF-expressing LGGs 1, 3 and 6 mpi. The lesions are outlined by the dotted lines. The percentage of the area occupied by the LGG is indicated within each panel. Scale bars, 200 μm. FIG. 5C contains representative Ki67 immunohistochemistry images (top panel) and bar graphs showing percentages of Ki67⁺ cells in Rag1^{-/-} mice harboring NF1-null or KIAA1549:BRAF-expressing iNPC glial or mixed/glial embryonal tumors at 1, 3 and 6 mpi. Scale bars, 200 μm. FIG. 5D contains images and bar graphs showing no change in TUNEL⁺ cells (apoptotic cells) in NF1-null and KIAA1549:BRAF-expressing iNPC-derived LGGs were observed 1 (0.41%; 0.38%; 0.38%), 3 (0.36%, 0.37%; 0.34%), and 6 months (0.38%, 0.37%; 0.38%) after injection. Scale bars, 100 μm. Data are represented as means ±SEM, one-way ANOVA with Bonferroni post-test correction, Individual p values are indicated within each graph. ns not significant.

FIG. 6 shows an exemplary embodiment of analysis of iNPC-injected Rag1^{-/-} mice in accordance with the present disclosure. FIG. 6 contains images showing there is no increase in beta-galactosidase⁺ (senescent) cells in Rag1^{-/-} brainstems injected with control (top panels; CTL; 0.3%) or 2041C>T^{-/-} and 6513T>A^{-/-} (NF1-null; lower panels; 0.3-0.4%) iNPCs at 1, 3 or 6 mpi. Scale bar is 100 μm.

FIG. 7 is a schematic showing differentiation of human iNPCs into iOPCs, iGRPs and astrocytes in accordance with the present disclosure.

FIG. 8(A-D) shows an exemplary embodiment of in vitro characterization of iGRPs, iOPCs, and astrocytes in accordance with the present disclosure. FIG. 8A contains images showing CTL and 6513T>A^{-/-} astrocytes are GFAP⁺, S100β⁺, EAAT1⁺ and EAAT2⁺ cells. FIG. 8B contains images and bar graphs showing CTL and 6513T>A^{-/-} iGRPs are CD133⁺, SOX2⁺, ABCG1⁺, O4⁺, GFAP⁺ and S100β⁺, but MBP^{neg} cells (top) and quantification of the percentage of GFAP- and MBP-immunopositive iGRPs (bottom). FIG. 8C contains images and bar graphs showing CTL and 6513T>A^{-/-} iOPCs are O4⁺, MBP⁺, GFAP⁺ and NG2^{neg} cells (top) and quantification of the percentage of GFAP- and MBP-immunopositive iOPCs (bottom). Data are shown as means ±SEM. FIG. 8D contains images showing iGRP- and iOPC-LGGs are immunonegative for non-specific pluripotency markers (OCT4 and Nanog) and endoderm and mesoderm markers (SMA and AFP), but immunopositive for the neural progenitor pluripotency marker Nestin. All scale bars, 100 μm.

FIG. 9 contains images showing immunostaining of KIAA1549:BRAF-expressing iGRP and iOPC LGGs in accordance with the present disclosure. H&E, Ku80, Ki67, GFAP, OLIG2, synaptophysin, BLBP, CD133, ABCG1, PDPN, SOX10 and P16 immunostaining images of KIAA1549:BRAF-expressing iGRP-(top) and iOPC- (bottom) LGGs at 1 mpi. Scale bar, 50 μm.

FIG. 10(A-E) is an exemplary embodiment showing iGRPs form LGGs in Rag1^{-/-} mice in accordance with the present disclosure. FIG. 10A is a table showing Rag1^{-/-} mice harbor LGGs at 1 mpi following CTL, 2041C>T^{-/-} and 6513T>A^{-/-} NF1-null iGRP and iOPC brainstem injections, as well as KIAA1549:BRAF-expressing iGRP and iOPC cerebellum injections. No LGGs were observed following NF1-null or KIAA1549:BRAF-expressing hiPSC-astrocyte injections. G, glial only; G/E, mixed embryonal/glial. FIG. 10B contains images showing analysis of 2041C>T^{-/-} NF1-null differentiated cells revealed CD133⁺, SOX2⁺, ABCG1⁺, O4⁺, GFAP⁺ and S100β⁺ iGRPs (top panel), GFAP⁺, S100b⁺, EAAT⁺ and EAAT2⁺ astrocytes (middle panel) and O49⁺, MBP⁺, GFAP⁺ and NG2^{neg} iOPCs (lower panel). FIG. 100 contains representative low-magnification H&E images of 2041C>T^{-/-} and 6513 T>A^{-/-} NF1-null and KIAA1549:BRAF-expressing iGRP and iOPC LGGs. Scale bars, 1 mm. FIG. 10D contains H&E, Ku80, Ki67, GFAP, OLIG2, synaptophysin, BLBP, CD133, ABCG1, PDPN, SOX10 and P16 immunostaining images of representative 2041C>T^{-/-} NF1-null iGRP- (top) and iOPC- (bottom) LGGs at 1 mpi. Scale bars, 100 μm. FIG. 10E contains a table and bar graph showing summary of relative immunopositivity scoring for GFAP, OLIG2, synaptophysin and Ki67 in LGG-bearing 1 mo mice. −, 0%; +, <25%; ++, 50%; +++, >75% immunopositive cells. Data are represented as means ±SEM, one-way ANOVA with Bonferroni post-test correction, p values are not significant.

FIG. 11 is a table showing mouse strains harboring NF1-null iNPC-derived LGGs at 1 mpi in accordance with the present disclosure.

FIG. 12 shows an exemplary embodiment of analysis of iNPC-injected mice in different genetically engineered mouse strains in accordance with the present disclosure. FIG. 12 contains representative images of H&E, CD3 (pan-T cell marker), Ku80, Ki67 and GFAP immunostaining of LGGs in Rag1^{-/-}, CD4-deficient, CD4/8-deficient, NOD/SCID mice, as well as H&E and CD3 staining of non-tumor-bearing wild type (VVT), CD8-deficient, and Cx3cr1^{-/-}; Ccr2^{-/-} mice one month after injection. Scale bars, 100 μm.

FIG. 13(A-B) is an exemplary embodiment showing CD4⁺ T cells control iNPC LGG formation in a Cxcl10-dependent manner in accordance with the present disclosure. FIG. 13A is a schematic detailing the experimental design. FIG. 13B is a heat map analysis of RNA sequencing performed on whole brainstem tissues from naive wild type (VVT) and Rag1^{-/-} mice showing segregation of transcript expression.

FIG. 14 shows an exemplary embodiment of analysis of iNPC-injected mice and RNA expression in different genetically engineered mouse strains in accordance with the present disclosure. FIG. 14 is a PCA plot of RNA sequencing performed on the brainstems of naïve WT and Rag1^{-/-} mice.

FIG. 15(A-B) is an exemplary embodiment showing CD4⁺ T cells control iNPC LGG formation in a Cxcl10-dependent manner in accordance with the present disclosure. FIG. 15A is a table showing list of >3-fold differentially expressed transcripts from the brainstems of Rag1^{-/-} mice relative to WT controls. FIG. 15B is a bar graph showing relative expression (R.E.) of Cxcl10 transcripts in WT and immunodeficient (CD8-deficient, NOD/SCID, CD4/8-deficient and CD4-deficient) mice. n=5; data are represented as means ±SEM; one-way ANOVA with Bonferroni post-test correction. Individual p values are indicated above each bar. ns, not significant.

FIG. 16 shows an exemplary embodiment of analysis of iNPC-injected mice and RNA expression in different genetically engineered mouse strains in accordance with the present disclosure. FIG. 16 contains bar graphs showing Relative expression (R.E.) of Chil3 and Cd59 in WT, CD8-deficient, Rag1^{-/-}, NOD/SCID, CD4/8-deficient, and CD4-deficient brainstem samples. Transcript expression is normalized to Gapdh expression. CTL, CD4/8-deficient mice, n=4; Rag1^{-/-} mice, n=5; NOD/SCID, CD4-deficient, CD8-deficient mice, each n=3 independently-generated samples. Data are represented as means ±SEM. Dashed lines indicate 0.5- and 1-fold relative expression.

FIG. 17(A-C) shows an exemplary embodiment of immunostaining of naïve mouse brainstems in accordance with the present disclosure. FIG. 17A and FIG. 17B contain images showing immunostaining of naïve (uninjected) mouse brainstems revealing unaltered microglial (lba1⁺) content (FIG. 17A), but reduced GFAP⁺, EAAT2⁺ and Aldh1|1⁺ cells (FIG. 17B) in mouse strains permissive of LGG formation (Rag1^{-/-}, NOD/SCID, CD4/8-deficient, CD4-deficient, and Cxcl10^{-/-} mice) relative to those that do not form LGGs (WT, CD8-deficient, and Cx3cr1^{-/-}; Ccr2^{-/-} mice). FIG. 17C contains bar graphs showing quantification of FIG. 17A-FIG. 17B. Data are represented as means ±SEM. One-way ANOVA with Bonferroni post-test correction. Individual p values are indicated within each graph. Scale bars, 100 μm.

FIG. 18(A-B) is an exemplary embodiment showing CD4⁺ T cells control iNPC LGG formation in a Cxcl10-dependent manner in accordance with the present disclosure. FIG. 18A is a bar graph showing relative Cxcl10 expression is reduced in Rag1^{-/-} astrocytes compared to WT controls. n=3; data are represented as means ±SEM, two-tailed student's t-test; p=0.0089. FIG. 18B contains a schematic of the experimental design and histogram demonstrating that Cxcl10 protein levels are increased by 6.5- and 24.4-fold in Rag1^{-/-} astrocytes treated with CD8⁺ and CD4⁺ T cell conditioned media (TCM), respectively, relative to untreated Rag1^{-/-} astrocytes. n=6; one-way ANOVA with Bonferroni post-test correction; individual p values are indicated above each bar.

FIG. 19(A-E) is an exemplary embodiment showing Cxcl10 absence is both necessary and sufficient for LGG formation in accordance with the present disclosure. FIG. 19A contains bar graphs showing NF1-null iNPC cell numbers decrease (direct cell count, top; 9-20% decrease) and the percent of cleaved caspase-3⁺ cells increases (bottom; 8.3-20.5-fold) following ectopic Cxcl10 expression or incubation with 25 or 100 pg/mL of recombinant Cxcl10 protein. FIG. 19B contains representative immunocytochemistry images and a bar graph demonstrating that ectopic Cxcl10 expression or treatment with increasing concentrations of recombinant Cxcl10 peptide induce an increase in GFAP⁺ astrocytic differentiation (Cxcl10: 8.3-fold, 25 pg/mL Cxcl10: 8.2-fold and 100 pg/mL Cxcl10: 20.5-fold increase), while 95-100% of the differentiated GFAP⁺ cells are undergoing apoptosis (cleaved caspase-3⁺) and 83.3-88.8% of the total number of cells undergoing apoptosis (cleaved caspase-3⁺) are GFAP⁺. FIG. 19C contains images showing (top) ectopic expression of murine Cxcl10 in 2041C>T^{-/-} NF1-null iNPCs and iGRPs (GFP expression, Western blot). α-tubulin was used as an internal protein loading control. (bottom) Ectopic murine Cxcl10 expression inhibits LGG formation in Rag1^{-/-} mice at 1 mpi (H&E images). Scale bar, 1 mm. FIG. 19D contains representative images of H&E, GFAP, OLIG2, synaptophysin and Ki67 staining of 2041C>T^{-/-} NF1-null iNPC- and iGRP- derived LGGs in Cxcl10^{-/-} mice at 1 mpi. The number of LGG-bearing mice is indicated within the H&E panels. Scale bars: left H&E panel, 1 mm; other panels, 100 μm. FIG. 19E is a bar graph denoting the percent of Ki67⁺ cells in the lesions. Data are represented as means ±SEM, (FIG. 19A-FIG. 19B) one-way ANOVA with Dunnett's multiple comparisons test, (FIG. 19E) two-tailed student's t-test. Individual p values are indicated within each graph. ns not significant.

FIG. 20(A-B) is an exemplary embodiment showing pediatric LGG cells form lesions in Rag1^{-/-} and Cxcl10^{-/-} mice in accordance with the present disclosure. FIG. 20A and FIG. 20B contain representative images of H&E, Ki67, GFAP and OLIG2 staining of pediatric LGG tumors in Rag1^{-/-} (top) and Cxcl10^{-/-} (bottom) mice at 1 mpi and 6 mpi using two pediatric LGG lines: JHH-NF-PA (FIG. 20A) and Res186 (FIG. 20B). The number of LGG-bearing mice is indicated within the H&E panels. Bar graphs denote the % Ki67⁺ cells in the lesions. Data are represented as means ±SEM. Scale bars: left H&E panel, 1 mm; other panels, 100 μm.

FIG. 21(A-B) is an exemplary embodiment showing PD0325901 treatment induces apoptosis and decreases cell proliferation in iNPC-LGGs in accordance with the present disclosure. FIG. 21A and FIG. 21B contain images and bar graphs showing TUNEL⁺ cells are increased (apoptotic cells; green; FIG. 21A), while Ki67⁺ cells are decreased (proliferating cells; green; FIG. 21B), in NF1^{-/-} iNPC-derived LGGs following PD0325901 treatment. Human tumor cells are Ku80⁺ (red; FIG. 21B). The % area occupied by the iNPC-LGGs is indicated within the images. Data are represented as means ±SEM, two-tailed student's t-test; Individual p values are indicated within each graph. Scale bars, 50 μm.

FIG. 22(A-B) shows an exemplary embodiment of in vitro treatment of iNPCs, iGRPs and iOPCs with PD0325901 in accordance with the present disclosure. FIG. 22A contains representative images showing PD0325901 treatment of isogenic control (CTL) or NF1-null iNPCs, iGRPs and iOPCs decreases proliferation (Ki67) and increases apoptosis (cleaved caspase-3). FIG. 22B contains bar graphs showing quantification of FIG. 22A. Data are represented as means ±SEM. 2-tailed student's t-test. Individual p values are indicated within each graph. ns, not significant. Scale bars, 100 μm.

FIG. 23(A-B) shows an exemplary embodiment of uncropped western blot images in accordance with the present disclosure. FIG. 23A is an uncropped immunoblot demonstrating increased phospho-ERK1/2Tyr^{202/204} relative to total ERK1/2 in KIAA1549:BRAF-iNPCs and isogenic controls (CTLs). FIG. 23B is an uncropped immunoblot demonstrating ectopic expression of murine Cxcl10 in 2041C>T^{-/-} iNPCs and iGRPs (GFP expression).

FIG. 24 is an exemplary embodiment showing pediatric LGG cells from pediatric pilocytic astrocytomas form tumors in Rag1^{-/-} mice in accordance with the present disclosure. FIG. 24 contains representative images of H&E, Ki67, and Neurofilament staining of the original pediatric LGG tumors (left) and pediatric LGG tumors in Rag1^{-/-} mice at 1 mpi (right) using two pediatric LGG lines: WUPA1, and WUPA2. The bar graph denotes the % Ki67⁺ cells (proliferative cells) in the tumors. Data are represented as means ±SEM.

### DETAILED DESCRIPTION

The present disclosure is based, at least in part, on the development of a humanized low-grade brain tumor model. As shown herein, it was discovered that by silencing Cxcl10 in mice, it is now possible to generate hiPSC progenitor-derived and patient-derived low-grade gliomas, which continue to grow in vivo for as long as 6 months.

One of the major barriers to identifying new therapies for pediatric brain tumors is the lack of human tumor models for preclinical discovery and evaluation. Using human induced pluripotent stem cells (hiPSCs) differentiated into neural progenitors, human pediatric low-grade gliomas in mice were developed, as described herein.

Moreover, leveraging several immune-defective mouse strains and RNA sequencing, it was discovered that astrocyte-produced CXCL10 is responsive for mediating tumor engraftment. By silencing Cxcl10 in mice, it is shown that it is now possible to generate hiPSC progenitor-derived and patient-derived low-grade gliomas, which continue to grow in vivo for as long as 6 months. This methodology has enabled the generation of low-grade gliomas in mice using human tumor lines that previously senesce within a week. This technology now sets the stage for precision medicine strategies in which patient-derived pediatric low-grade gliomas can now be grown in mice for drug testing.

This is believed to be the first model of pediatric low-grade gliomas, the most common brain tumor seen in children. The lack of these models has constituted a significant barrier to developing innovative therapies for these tumors.

Low grade glioma (LGG) is a major pediatric brain tumor that lacks good preclinical models (aside from in vitro systems) for the testing of novel diagnostics or therapeutics. The present invention disclosure describes mice with certain gene deletions that will be amenable to tumor transplantation models involving patient-derived xenograft (PDX) or cell-derived xenograft (CDX). To achieve favorable LGG growth in both the central or peripheral nervous system, the mice involved either must be homozygous Rag1⁻deficient or Cxc/10-deficient. Because the maintenance of immune signaling within the tumor microenvironment is necessary for LGG growth, the Rag1 ^{-/-} mice is considered an inferior host compared to the Cxcl10^{-/-} mice. Specifically, the former mutation prevents the maturation of B and T lymphocytes and as such, lacks the immunocompetence of the Cxcl10^{-/-} mutation.

LGG tumors for PDX studies can be collected from patient donors; examples include JHH-NF-PA and Res186 tumor lines, both of which have been successfully tested in the disclosed mouse models. LGG-like tumors for CDX studies can be derived from neuronal lineage iPSCs (called iNPCs) either by mutating the NF1 locus (c.2041C>T; c.6513T>A) or expressing the KIAA1549:BRAF fusion gene in situ. The transplanted iPSCs can be further differentiated into separate glial lineages: iGRP, iOPC or astrocytes, but only iGRP and iOPC exhibit LGG-like tumor growth.

Despite being the most prevalent brain tumor in children, pediatric low-grade gliomas have a dearth of preclinical models available, thus hindering drug development. PDX LGGs implanted into mice often have difficulty growing because of premature senescence and dependence on supportive immune environment. PDX LGG models that have been developed are sub-optimal because they carry genetic mutations that help implanted LGGs evade premature senescence but these mutations are uncharacteristic of actual childhood gliomas.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity, when necessary, through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:

(i) By disabling repressors. The gene is expressed because an inducer binds to the repressor. The binding of the inducer to the repressor prevents the repressor from binding to the operator. RNA polymerase can then begin to transcribe operon genes.

(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=XN100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6 X SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na^{'0}])+0.41(fraction G/C content)−0.63(% formamide)−(600/1). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Genome Editing

As described herein, Cxcl10 signals can be modulated (e.g., reduced, eliminated, or enhanced) using genome editing. Processes for genome editing are well known; see e.g., Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of Cxcl10 by genome editing can result in the ability for an animal to grow tumors recapitulating low grade glioma (LGG) tumors.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for the removal or addition of Cxcl10 signals (e.g., activate (e.g., CRISPRa), upregulate, overexpress, downregulate Cxcl10).

For example, the methods as described herein can comprise a method for altering a target polynucleotide sequence in a cell comprising contacting the polynucleotide sequence with a clustered regularly interspaced short palindromic repeats-associated (Cas) protein.

Gene Therapy and Genome Editing

Gene therapies can include inserting a functional gene with a viral vector. Gene therapies for cancers are rapidly advancing.

There has recently been an improved landscape for gene therapies. For example, in the first quarter of 2019, there were 372 ongoing gene therapy clinical trials (Alliance for Regenerative Medicine, May 9, 2019).

Any vector known in the art can be used. For example, the vector can be a viral vector selected from retrovirus, lentivirus, herpes, adenovirus, adeno-associated virus (AAV), rabies, Ebola, lentivirus, or hybrids thereof.

### Gene Therapy Strategies

| | |
|---|---|
| | Associated experimental |
| Strategy | models |

Viral Vectors

| | | |
|---|---|---|
| Retroviruses | Retroviruses are RNA viruses | Murine model of MPS VII |
| | transcribing their single-stranded | Canine model of MPS VII |
| | genome into a double-stranded | |
| | DNA copy, which can integrate | |
| | into host chromosome | |
| Adenoviruses (Ad) | Ad can transfect a variety of | Murine model of Pompe, Fabry, |
| | quiescent and proliferating | Walman diseases, |
| | cell types from various species | aspartylglucosaminuria |
| | and can mediate | and MPS VII |
| | robust gene expression | |
| Adeno-associated | Recombinant AAV vectors | Murine models of Pompe, Fabry |
| Viruses (AAV) | contain no viral DNA and can | diseases, Aspartylglucosaminuria, |
| | carry ~4.7 kb of foreign | Krabbe disease, Metachromatic |
| | transgenic material. They | leukodystrophy, MPS I, MPSII, |
| | are replication defective and can | MPSIIIA, MPSIIIB, MPSIV, |
| | replicate only while | MPSVI, MPS VII, CLN1, CLN2, |
| | coinfecting with a helper virus | CLN3, CLN5, CLN6 |

Non-viral vectors

| | | |
|---|---|---|
| plasmid DNA | pDNA has many desired | Mouse model of Fabry disease |
| (pDNA) | characteristics as a gene | |
| | therapy vector; there are no limits | |
| | on the size or genetic | |
| | constitution of DNA, it is relatively | |
| | inexpensive to supply, | |
| | and unlike viruses, antibodies are | |
| | not generated against DNA in normal | |
| | individuals | |
| RNAi | RNAi is a powerful tool for gene | Transgenic mouse strain |
| | specific silencing that | Mouse models of acute liver |
| | could be useful as an enzyme | failure |
| | reduction therapy or | Mice with hepatitis B virus |
| | means to promote read-through | Fabry mouse |
| | of a premature stop codon | |

Gene therapy can allow for the constant delivery of the enzyme directly to target organs and eliminates the need for weekly infusions. Also, correction of a few cells could lead to the enzyme being secreted into the circulation and taken up by their neighboring cells (cross-correction), resulting in widespread correction of the biochemical defects. As such, the number of cells that must be modified with a gene transfer vector is relatively low.

Genetic modification can be performed either ex vivo or in vivo. The ex vivo strategy is based on the modification of cells in culture and transplantation of the modified cell into a patient. Cells that are most commonly considered therapeutic targets for monogenic diseases are stem cells. Advances in the collection and isolation of these cells from a variety of sources have promoted autologous gene therapy as a viable option.

The use of endonucleases for targeted genome editing can solve the limitations presented by the usual gene therapy protocols. These enzymes are custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating, preventing, or reversing cancer (e.g., LGG) in a subject in need of administration of a therapeutically effective amount of an agent, so as to substantially inhibit cancer, slow the progress of cancer, or limit the development of cancer.

Methods described herein are generally performed on a subject in need thereof. A subject in need of the therapeutic methods described herein can be a subject having, diagnosed with, suspected of having, or at risk for developing cancer. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of an agent is, for example, an amount that would cause the desired therapeutic effect in a subject while minimizing undesired side effects. In various embodiments, an effective amount of an agent described herein can substantially inhibit cancer, slow the progress of cancer, or limit the development of cancer.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of an agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to substantially inhibit cancer, slow the progress of cancer, or limit the development of cancer.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Shamel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of an agent can occur as a single event or over a time course of treatment. For example, an agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for cancer.

An agent can be administered simultaneously or sequentially with another agent, such as an antibiotic, an anti-inflammatory, or another agent. For example, an agent can be administered simultaneously with another agent, such as an antibiotic or an anti-inflammatory. Simultaneous administration can occur through administration of separate compositions, each containing one or more of an agent, anti-cancer agent, an antibiotic, an anti-inflammatory, or another agent. Simultaneous administration can occur through administration of one composition containing two or more of an agent, anti-cancer agent, an antibiotic, an anti-inflammatory, or another agent. An agent can be administered sequentially with an anti-cancer agent, an antibiotic, an anti-inflammatory, or another agent. For example, an agent can be administered before or after administration of an anti-cancer agent, an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models is also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

Cell Therapy

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogeneic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 pm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

Screening

Also provided are screening methods using the models and cells as described herein.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MVV) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about -2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and 0 atoms). Also, drug-like molecules typically have a span (breadth) of between about 8A to about 15A.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to animals, test agents, cell lines, etc. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

### Human Induced Pluripotent Stem Cell Engineering Establishes a Humanized Mouse Platform for Pediatric Low-Grade Glioma Modeling

This example describes the development of a humanized low-grade brain tumor model.

Abstract

A major obstacle to identifying improved treatments for pediatric low-grade brain tumors (gliomas) is the inability to reproducibly generate human xenografts. To surmount this barrier, human induced pluripotent stem cell (hiPSC) engineering was leveraged to generate low-grade gliomas (LGGs) harboring the two most common pediatric pilocytic astrocytoma-associated molecular alterations, NF1 loss and KIAA1549:BRAF fusion. Herein is identified that hiPSC-derived neuroglial progenitor populations (neural progenitors, glial restricted progenitors, and oligodendrocyte progenitors), but not terminally differentiated astrocytes, give rise to tumors retaining LGG histologic features for at least 6 months in vivo. Additionally, it was demonstrated that hiPSC-LGG xenograft formation requires the absence of CD4 T cell-mediated induction of astrocytic Cxcl10 expression. Genetic Cxcl10 ablation is both necessary and sufficient for human LGG xenograft development, which additionally enables the successful long-term growth of patient-derived pediatric LGGs in vivo. Lastly, MEK inhibitor (PD0325901) treatment increased hiPSC-LGG cell apoptosis and reduced proliferation both in vitro and in vivo. Collectively, this study establishes a tractable experimental humanized platform to elucidate the pathogenesis of and potential therapeutic opportunities for childhood brain tumors.

Introduction

Low-grade gliomas (LGGs; World Health Organization grade 1 and 2 astrocytomas) are the most frequently occurring cancers of the central nervous system (CNS) in children, accounting for one third of all pediatric brain tumors. The most common pediatric LGG is the grade 1 pilocytic astrocytoma (PA), which arises sporadically or in the context of the neurofibromatosis type 1 (NF1) tumor predisposition syndrome. In contrast to gliomas in adults, pediatric LGGs are typically slow-growing neoplasms located in the cerebellum, brainstem, and optic pathway, with an overall 10-year survival rate of >90%. Importantly, PAs in children rarely progress to high-grade malignancy and infrequently result in death. As such, pediatric LGGs represent a chronic condition that causes significant life-long morbidity, including vision loss, neurologic deficits, and endocrine complications.

While preclinical mouse and pig models of NF1-associated optic pathway glioma (NF1-OPG) and BRAF-driven sporadic pediatric LGGs have helped elucidate the pathobiology of these tumors and served as platforms for therapeutic drug testing, they only partially capture the essential properties of their human counterparts. Unfortunately, efforts to develop patient-derived pediatric LGG xenograft (PDX) models have been hindered by multiple factors, including their intrinsic slow growth rates, propensity to undergo premature senescence, low clonogenic frequency, and heavy dependence on a supportive microenvironment. In addition, the few existing human pediatric LGG xenograft models harbor genetic mutations (TP53/ CDKN1A or CDKN2A/RB1 alterations) uncharacteristic of these childhood gliomas, especially PAs, which were specifically introduced to permit pediatric LGG cells to escape cellular senescence. Herein, human induced pluripotent stem cell (hiPSC) engineering was leveraged to generate humanized NF1-associated and sporadic KIAA1549:BRAF-driven pediatric LGG models, identify potential cells of origin for these tumors, and discover a CD4⁺ T cell-astrocyte Cxcl10 axis critical for LGG xenograft formation. Disruption of Cxcl10 expression established a new murine platform for in vivo patient-derived pediatric LGG xenograft modeling and therapeutic evaluation.

Results

NF1 -Null and KIAA1549:BRAF-Expressing Human Induced Pluripotent Stem Cell (hiPSC)-Derived Neural Progenitor Cells (iNPCs) Exhibit Increased Cell Proliferation

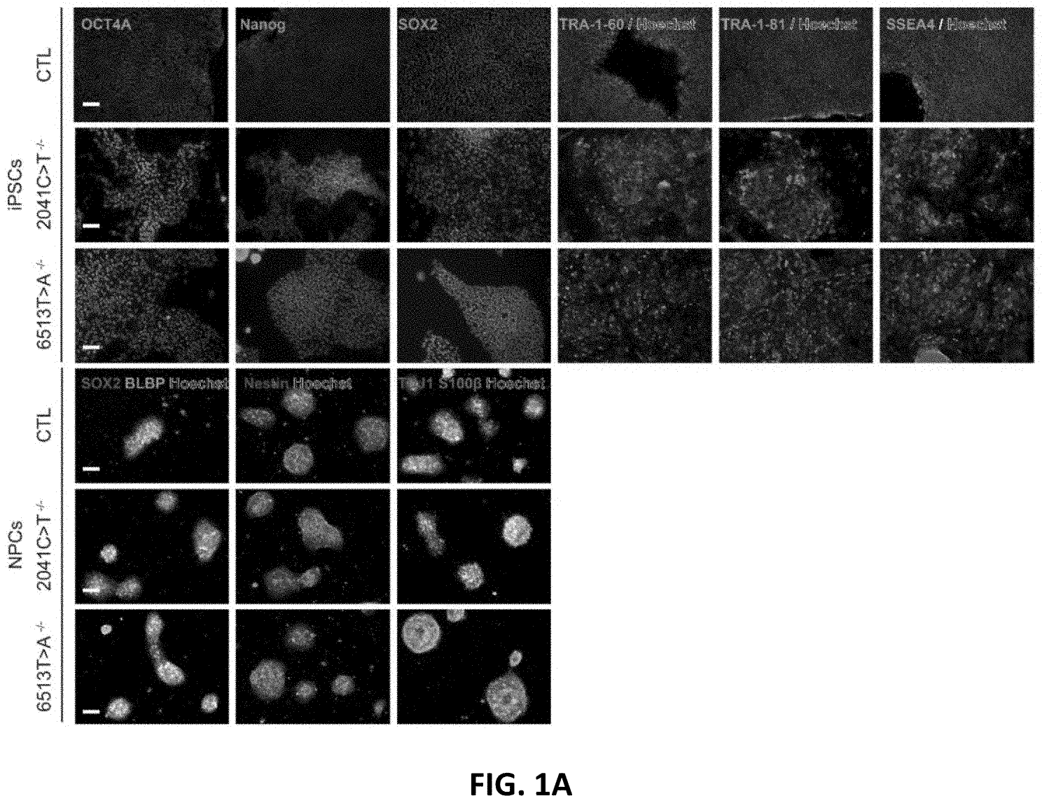

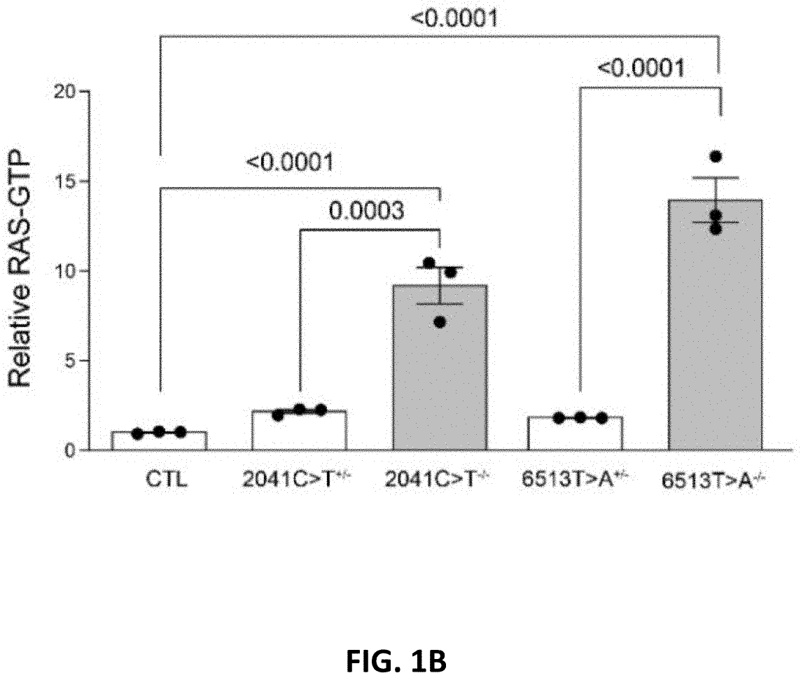

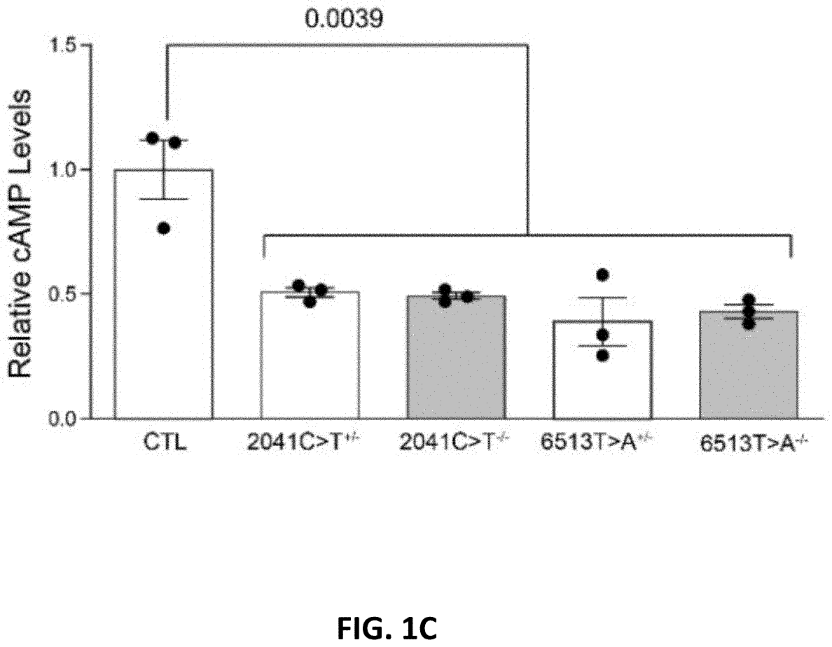

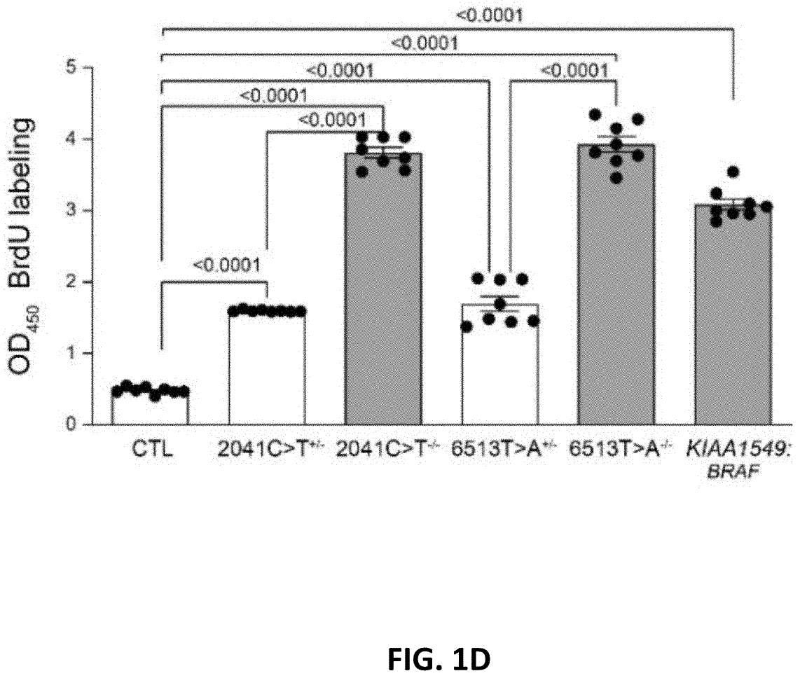

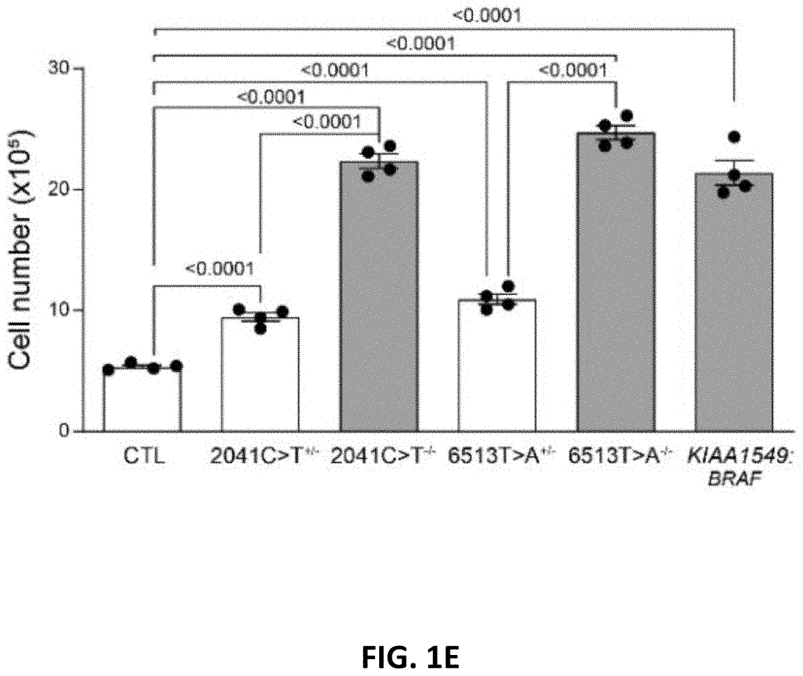

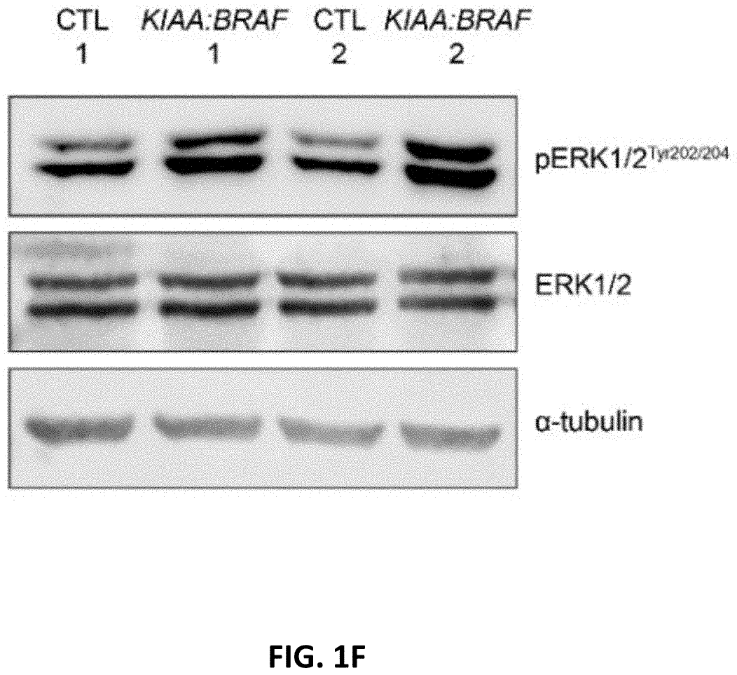

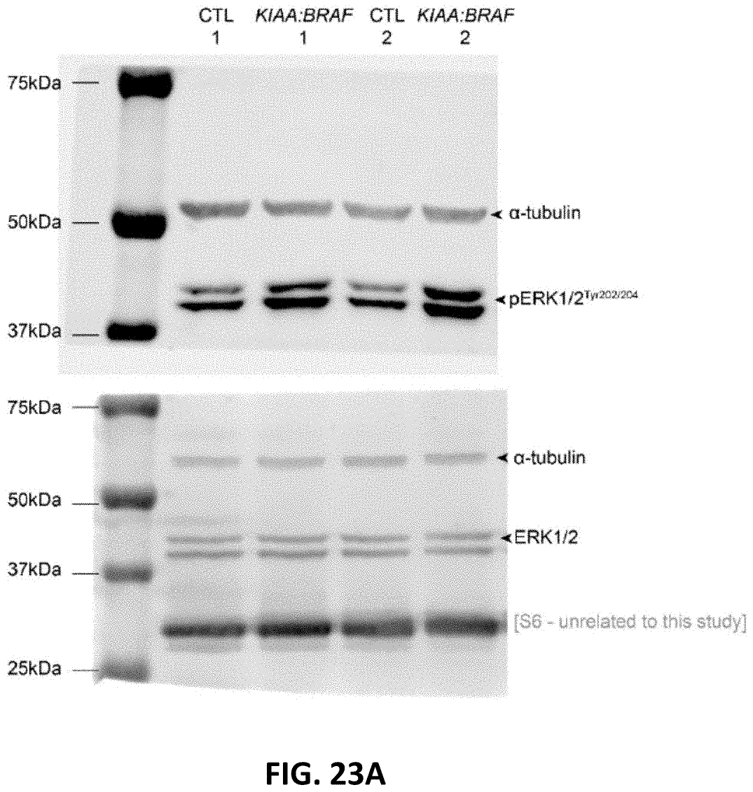

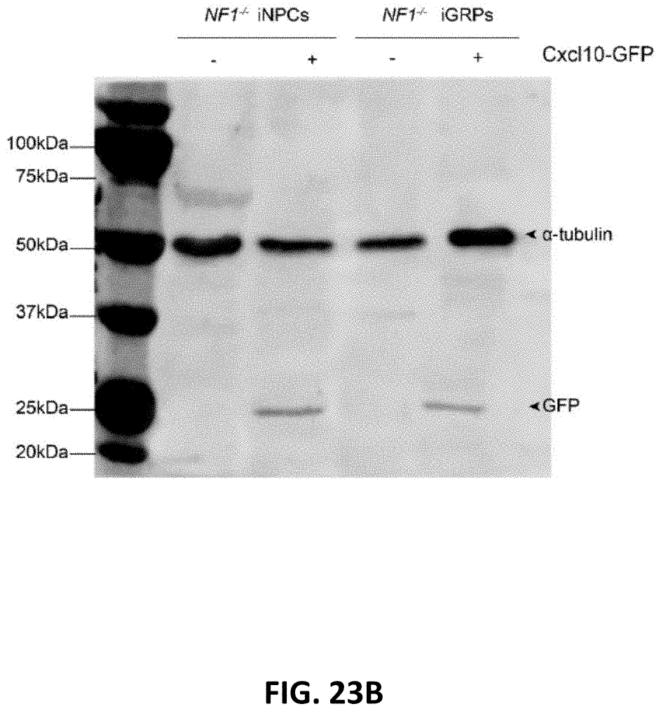

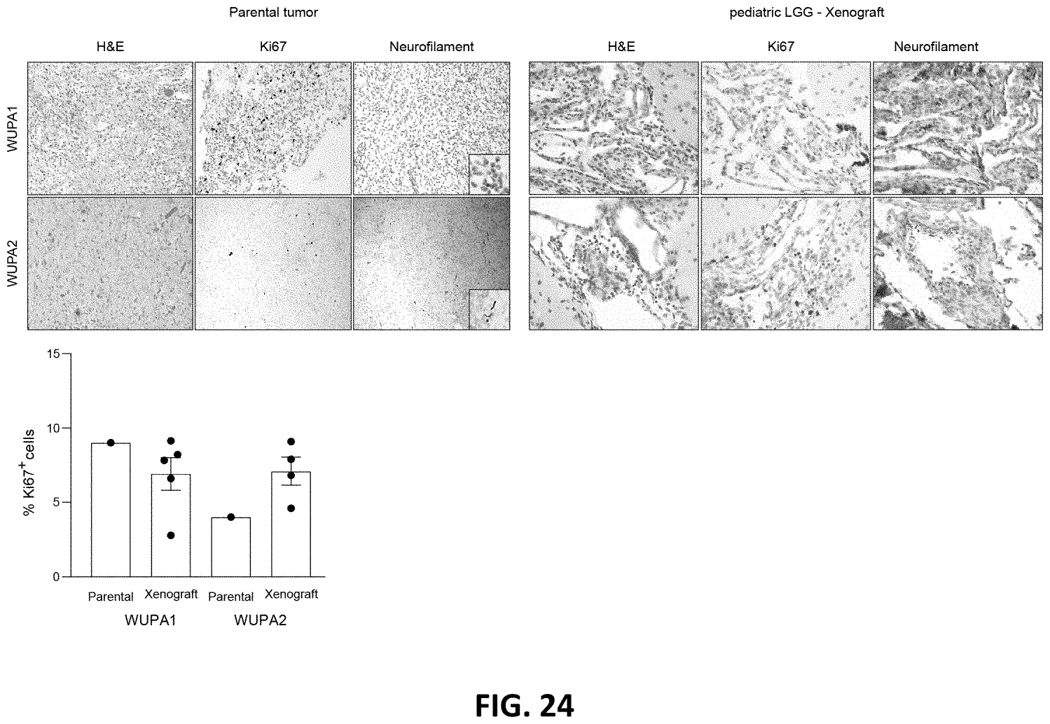

To model NF1 loss of heterozygosity in pediatric LGGs arising in the NF1 brain tumor predisposition syndrome, two different hiPSCs lines were engineered, each with a distinct homozygous germline NF1 patient-derived NF1 mutation (c.2041C>T^{-/-} and c.6513T>A^{-/-}; see e.g., FIG. 1A). Although the cell of origin for human pediatric LGGs is currently unknown, murine Nf1 LGGs of the optic nerve/chiasm (optic pathway gliomas; OPGs) arise from neural stem or neuroglial (progenitor) cells. As such, NF1-null (c.2041C>T^{-/-} and c.6513T>A^{-/-}) and control (isogenic iPSCs that have undergone identical CRISPR/ Cas9 processing as the NF1-null iPSC lines, but without the introduction of a mutation) hiPSCs were first differentiated into multipotent human neural stem cells (iNPCs) capable of generating both neuronal and glial lineage cells (see e.g., FIG. 1A). Consistent with the established role of the NF1 protein, neurofibromin, as a negative RAS regulator, these NF1-null iNPCs had increased RAS activity (2041C>T^{-/-} 9.2-fold; 6513T>A^{-/-} 13.9-fold; 2041C>T^{+/-} 2.2-fold⁻; 6513T>A^{+/-}, 1.8-fold relative to CTL iNPCs; see e.g., FIG. 1B), but similar cAMP levels (2041C>T^{-/-}, 51% reduction; 6513T>A^{-/-}, 57% reduction; 2041C>T^{+/-}, 49% reduction; 6513T>A^{+/-}, 61% reduction; see e.g., FIG. 1C), relative to NF1-mutant iNPCs heterozygous for the same germline NF1 mutations, as previously reported in mice. In addition, to model sporadic pediatric LGGs resulting from genomic rearrangements involving the BRAF kinase gene, KIAA1549:BRAF-expressing iNPCs were generated. Both NF1-null and KIAA1549:BRAF-expressing iNPCs had increased proliferation, as measured by BrdU incorporation (2041C>T^{-/-}, 7.9- fold; 6513T>8.2-fold; 2041C>Ti^{+/-}, 3.3-fold^{+/-}; 6513T>A^{-/-}, 3.5-fold; KIAA1549:BRAF, 7.5-fold) and direct cell counting (2041C>T^{-/-}, 4.2- fold; 6513T>A^{-/-}, 4.6-fold; 2041C>T^{+/-}, 1.8-fold⁻; 6513T>A^{+/-}, 2-fold; KIAA1549:BRAF, 3.1-fold), relative to control iNPCs (see e.g., FIG. 1D-FIG. 1E). Moreover, KIAA1549:BRAF-expressing iNPCs demonstrated increased phosphorylation of ERK1/2 relative to their isogenic controls, indicative of increased MAPK pathway activation (see e.g., FIG. 1F and FIG. 23A-FIG. 23B).

iNPCs form LGGs in Rag1^{-/-} Mice

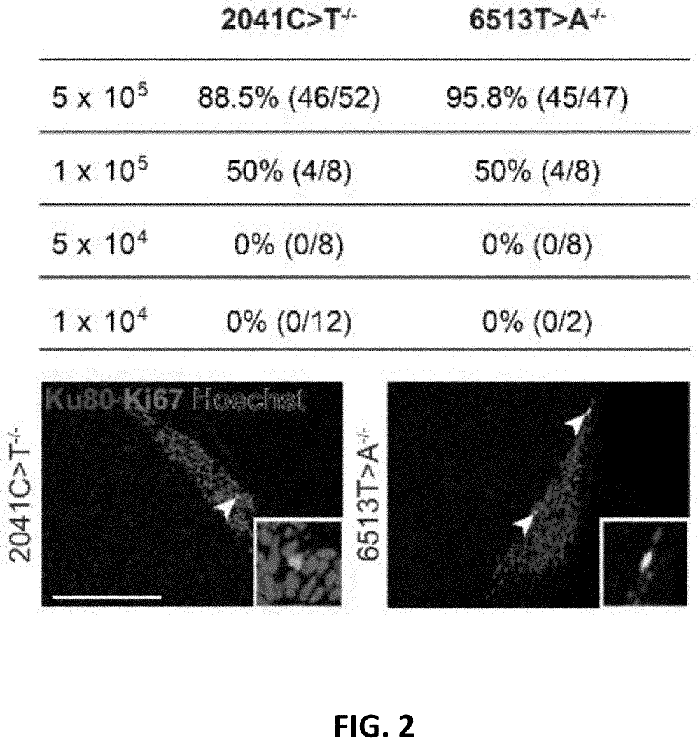

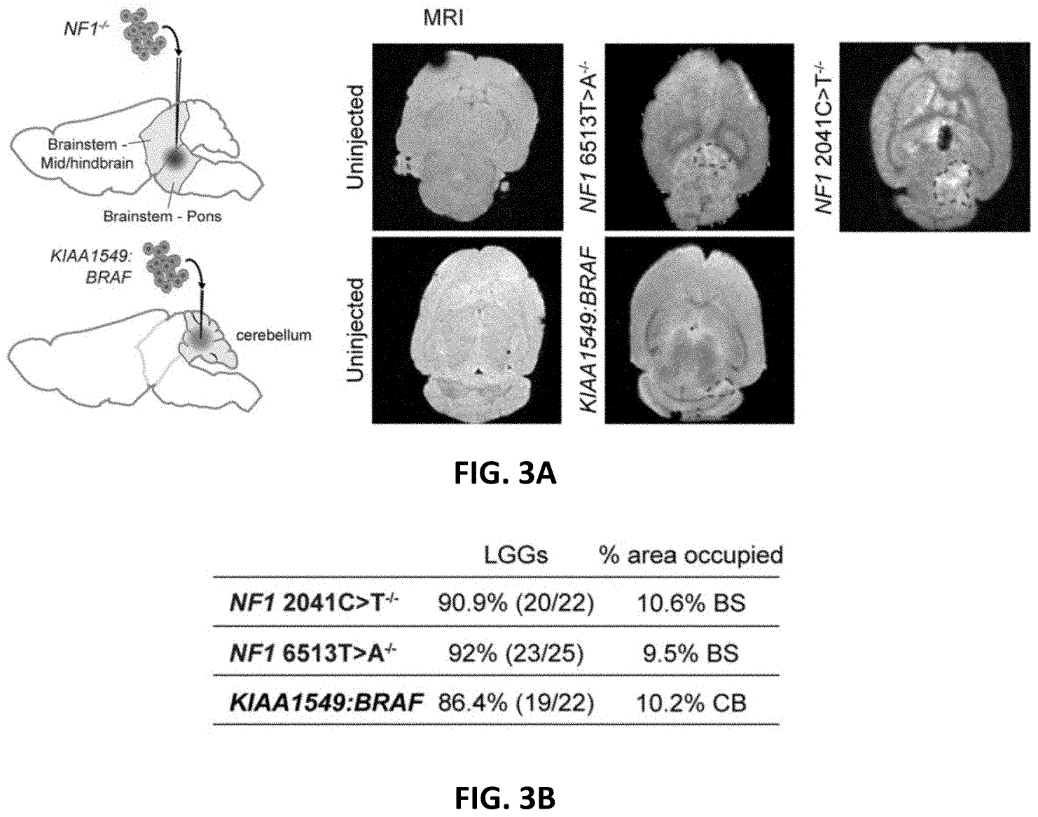

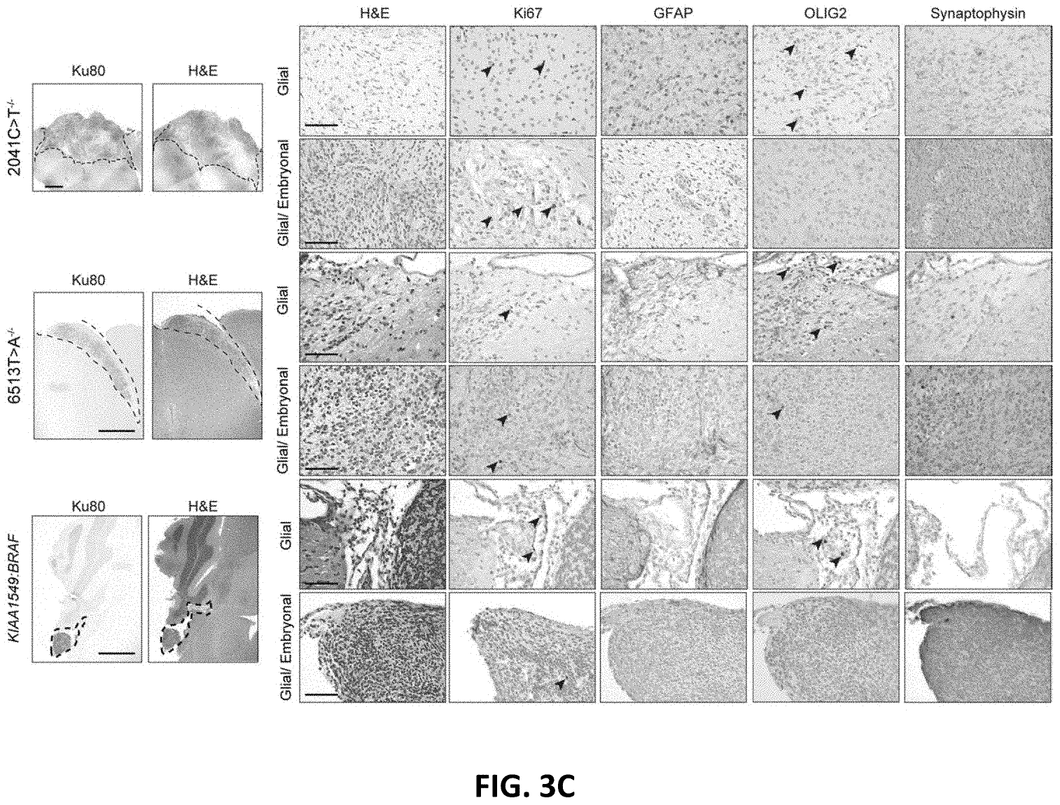

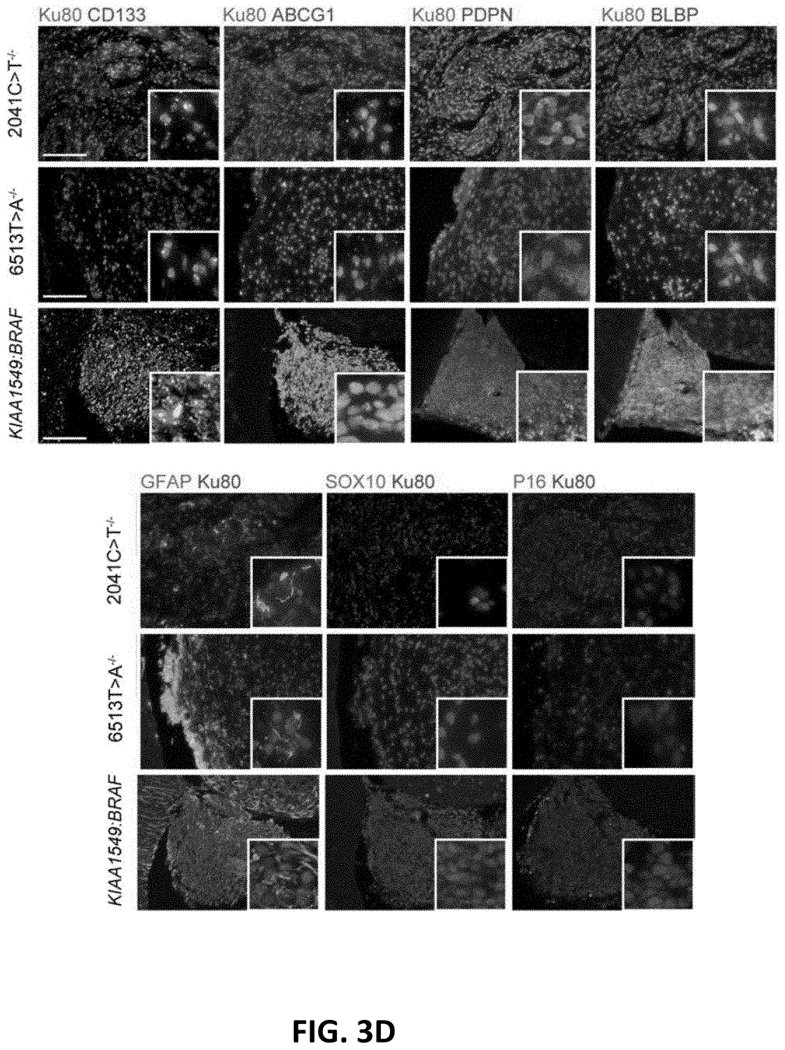

To determine whether NF1-null iNPCs generate LGGs in immunocompromised mice, 1×10⁴ to 5×10⁵ CTL or NF1-null iNPCs were implanted into the brainstems of Rag1^{-/-} mice at 0-3 days of age (PN0-3). The brainstem was chosen for several reasons: (1) it is the second most common brain location for pediatric LGGs arising in children with NF1, (2) brainstem injections were previously used for murine Nf1-OPG stem cell tumor modeling, and (3) injections into the mouse optic nerve, the most common site for NF1- pediatric LGGs, create major tissue damage and induce a reactive immune microenvironment. Using established neuropathological criteria for pediatric LGGs, a LGG was defined as (1) a mass-occupying lesion with architectural distortion by standard H&E staining (and by MRI in a subset of cases), with (2) increased proliferation (Ki67 labeling index>1%) and (3) immunopositivity for glial immunohistochemical markers used in the routine diagnosis of human low-grade gliomas (e.g., GFAP and OLIG2). Based on these criteria and leveraging this hiPSC/murine platform, both NF1-null iNPC lines (2041C>T^{-/-}, 6513T>A^{-/-}) formed LGGs 1 month post-injection (mpi) in approximately 50% of mice injected with 1×10⁵ iNPCs and in >85% of animals injected with 5×10⁵ iNPCs (see e.g., FIG. 2). Similarly, to model sporadic pediatric LGGs, 5×10⁵ KIAA1549:BRAF-expressing iNPCs were orthotopically transplanted into the cerebella of Rag1^{-/-} mice, the most common location for sporadic PA with this molecular alteration. Over 85% of all Rag1^{-/-} mice injected with KIAA1549:BRAF-expressing iNPCs formed LGGs at 1 mpi (see e.g., FIG. 3A-FIG. 3B). LGG sections were reviewed by an expert human neuropathologist.

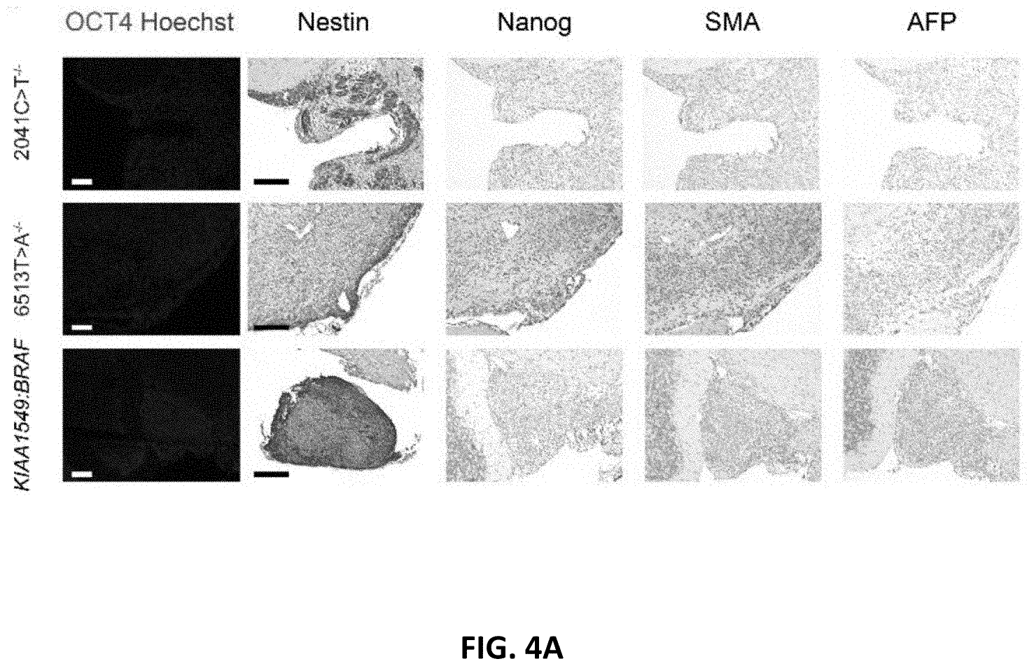

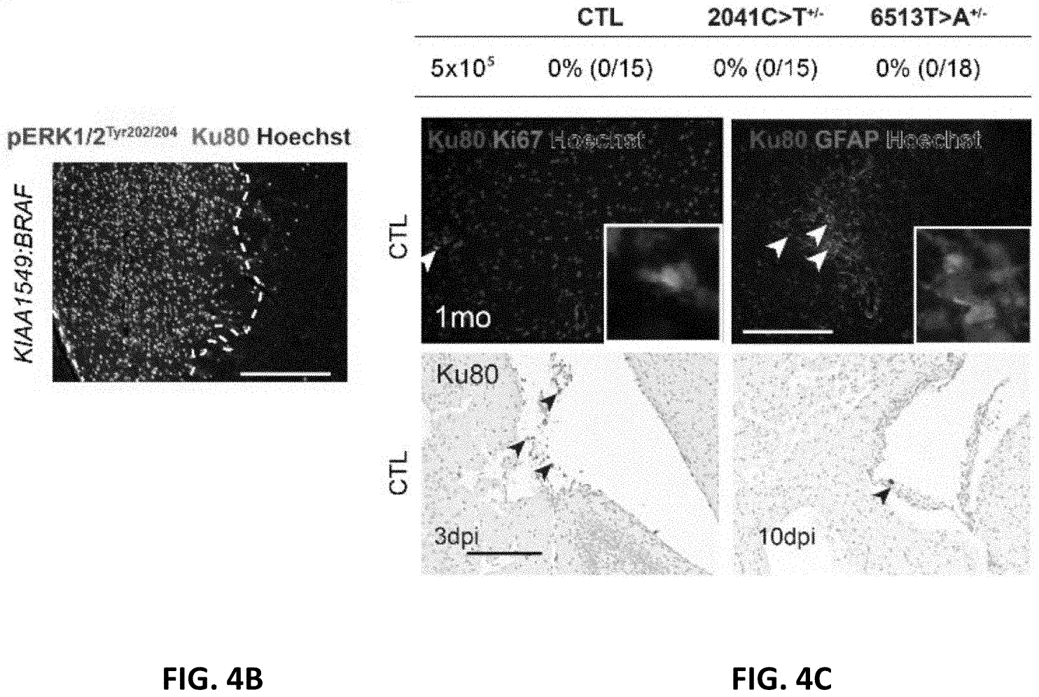

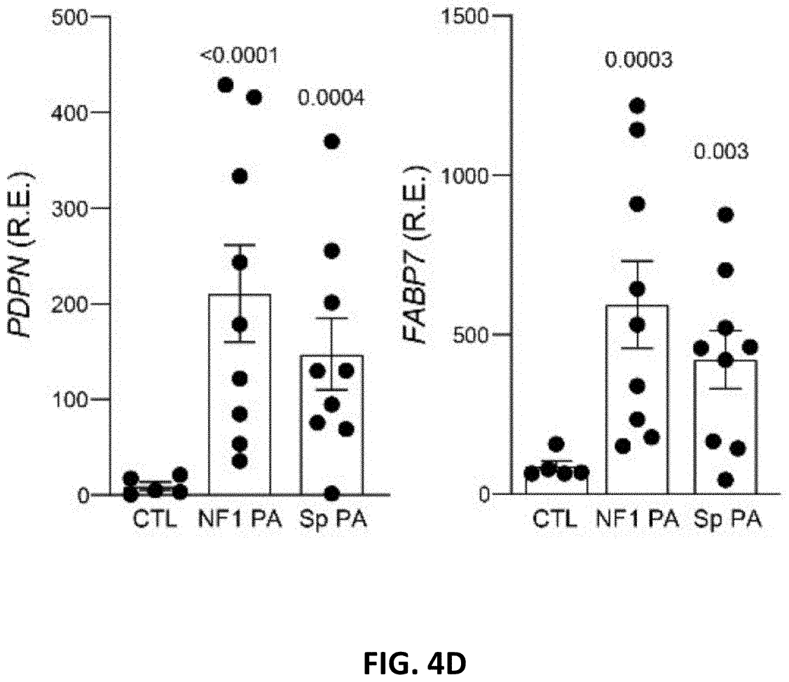

NF1-associated and KIAA1549:BRAF-driven lesions were also detectable by magnetic resonance imaging (4.7-Tesla MRI, see e.g., FIG. 3A) and exhibited many of the histopathologic features of human pediatric LGGs. These tumors were composed of human cells (Ku80⁺ cells), were hypercellular, mostly parenchymal with exophytic components, either anterior or lateral to the midbrain/brainstem tissue (NF1-null iNPC tumors) or anterior to the cerebellum (KIAA1549:BRAF-expressing iNPC tumors), and well-circumscribed (see e.g., FIG. 3C). The tumors contained GFAP- and OLIG2-immunopositive cells (see e.g., FIG. 3C), as seen in pediatric LGGs. All of the iNPC-lesions contained both glial areas, as determined by H&E, GFAP and OLIG2 (glial) immunopositivity, and embryonal-like hypercellular areas with neuronal (synaptophysin⁺) components, some of which contained neuroepithelial rosettes. Since the resulting tumors did not exhibit some histologic features routinely observed in pilocytic astrocytomas (eosinophilic granular bodies, Rosenthal fibers), they were classified as LGGs, which is the routine diagnostic approach in clinical practice. Moreover, the Ku80⁺ cells in these LGGs also expressed CD133 and ABCG1 (see e.g., FIG. 3D), markers of glioma stem cells, but were immunonegative for SOX10 and p16 expression, which can be observed in some patient pediatric LGGs (see e.g., FIG. 3D). iNPC LGGs were negative for the OCT4 and NANOG hiPSC pluripotency markers, as well as for SMA and AFP endoderm and mesoderm markers, but were immunopositive for the Nestin neural stem cell marker (see e.g., FIG. 4A). Additionally, KIAA1549:BRAF-expressing LGGs demonstrated increased ERK1/2 activity relative to the surrounding non-neoplastic brain tissue (see e.g., FIG. 4B). Importantly, neither heterozygous NF1-mutant nor control iNPCs formed LGGs in Rag1^{-/-} mice (see e.g., FIG. 4C), consistent with the requirement for NF1 loss of heterozygosity in NF1-LGG tumorigenesis.

RNA sequencing or methylation studies were not used to compare the humanized iNPC-LGGs with resected patient LGGs: iNPC-LGGs are composed entirely of human neoplastic cells and cannot be directly compared with human LGG biospecimens, in which 30-50% of the cells are monocytes, neurons, and T cells. In addition, while methylation has proven valuable for separating classes of brain tumors, it is not as accurate in distinguishing subclasses of pediatric LGGs and glioneuronal tumors. This is due in part to the variable contributions of non-neoplastic elements, with the diagnostic standard involving histology, immunohistochemistry, and genetic analysis for the specific drivers involved (e.g., BRAF, NF1). For these reasons, two additional complementary approaches were employed to compare hiPSC-LGGs to their spontaneously arising clinical counterparts. First, leveraging human bulk RNA sequencing data, PDPN was identified as a gene differentially expressed both in NF1-associated and sporadic pilocytic astrocytomas relative to control non-neoplastic brain tissue (see e.g., FIG. 4D). Second, FABP7, which was previously shown to be overexpressed in mouse optic gliomas relative to control optic nerve by bulk RNA sequencing, was increased both in NF1-associated and sporadic pediatric LGGs relative to control non-neoplastic human brain (see e.g., FIG. 4D). Similar to their spontaneously arising pediatric LGG counterparts, both PDPN and BLBP (FABP7 protein) were expressed in the humanized NF1-null and KIAA1549:BRAF-associated LGGs in situ (see e.g., FIG. 3D).

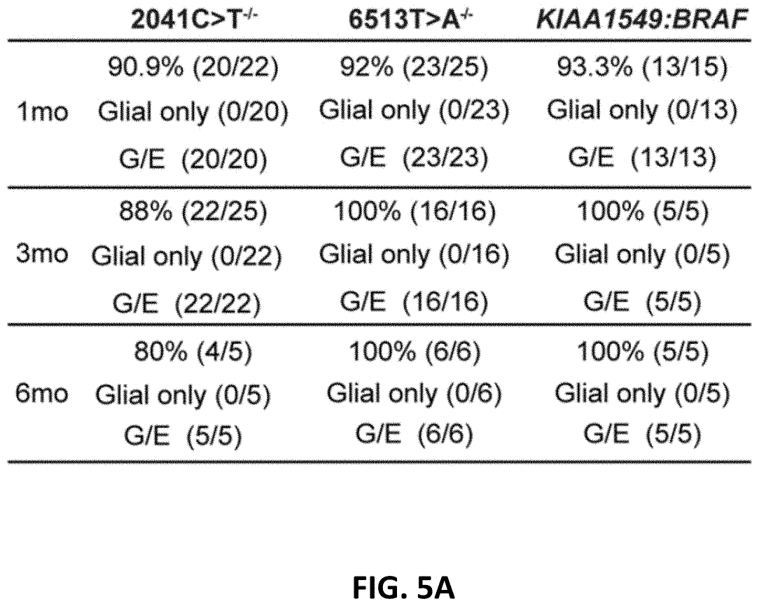

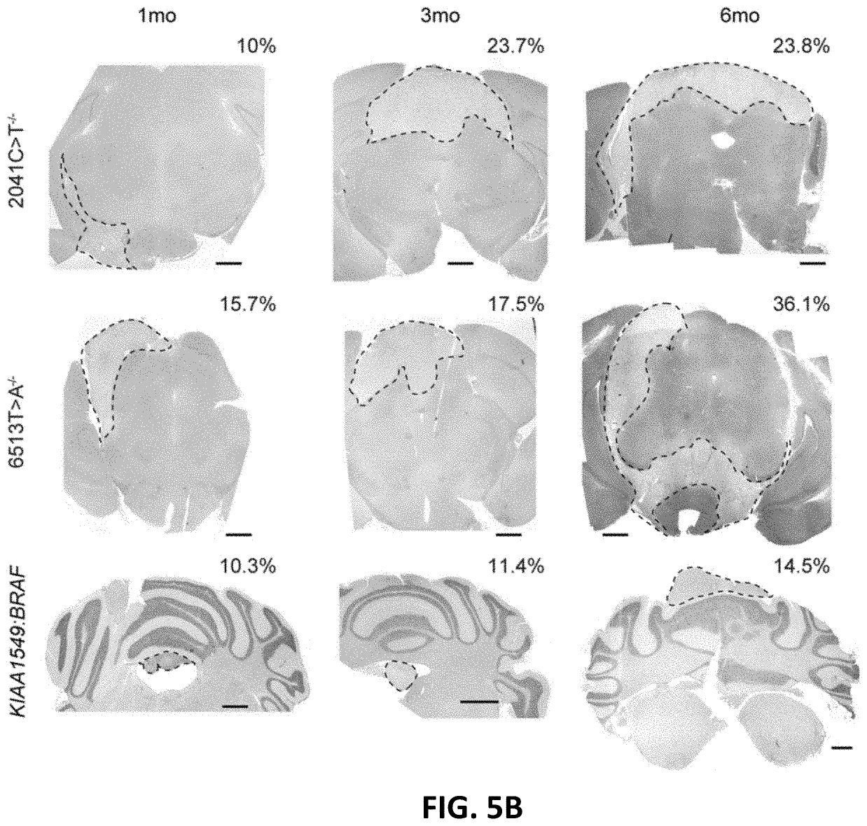

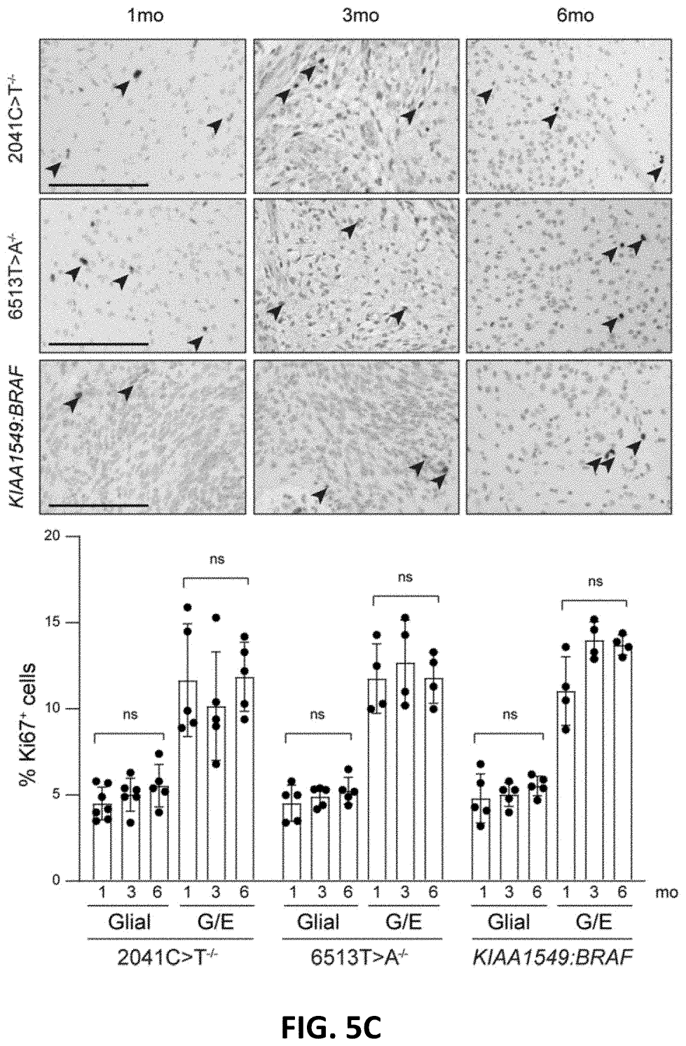

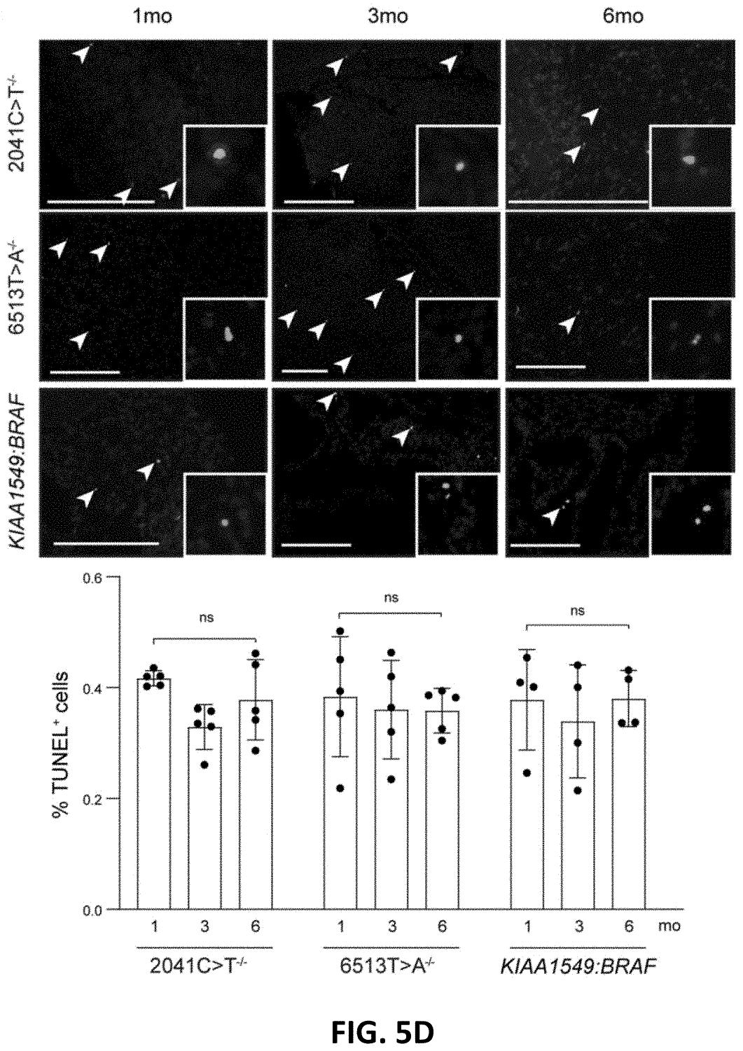

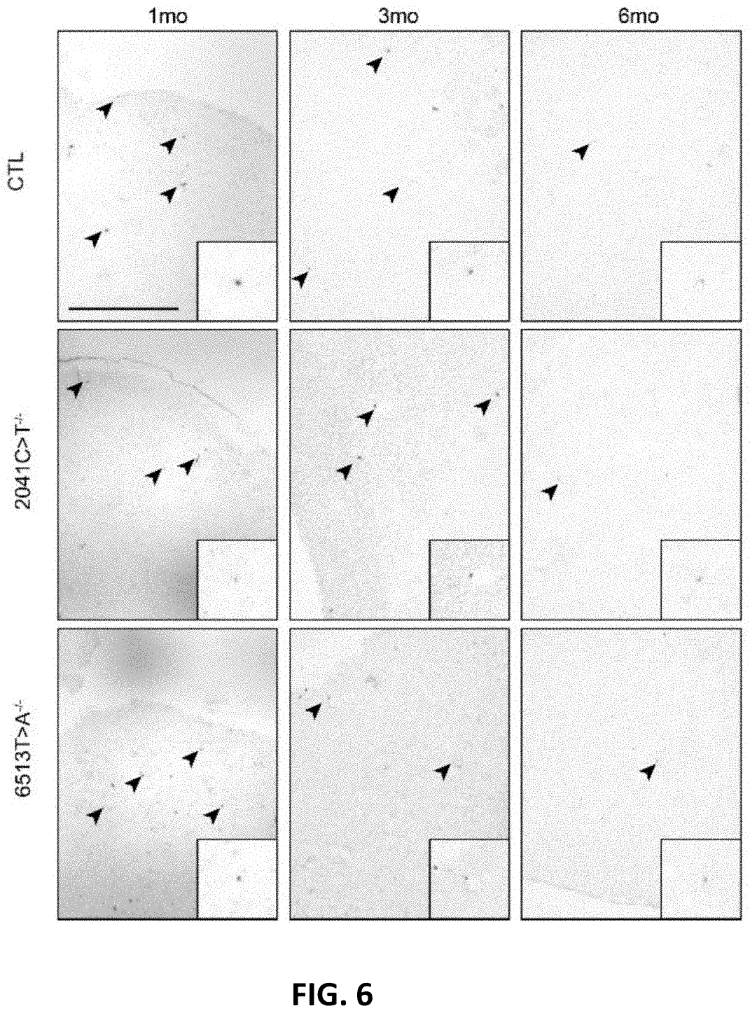

As expected for pediatric low-grade tumors, and mirroring clinical observations, mice with hiPSC-derived LGGs did not exhibit increased mortality nor obvious abnormal neurologic findings. To determine whether these lesions reflected the chronic non-malignant nature of pediatric LGGs, tumors were assessed at 1, 3 and 6 mpi (see e.g., FIG. 5A-FIG. 5D). Using power calculations with a two-sided, two portions test, where power was set at 80% and significance at 0.05, it was estimated that a minimum of 4, 4, and 5 mice would be required to detect tumor formation at 1 mpi, 3 mpi and 6 mpi, respectively. With appropriately powered cohorts, mice injected with NF1-null and KIAA1549:BRAF-expressing iNPCs formed LGGs that were histologically similar at 1, 3 and 6 mpi (see e.g., FIG. 5A). While these lesions grew in size over time, occupying on average 10-36% of the brainstem or 10-15% of the cerebellum, respectively (see e.g., FIG. 5B), they had similar proliferative indices (Ki67⁺ cells) at 1, 3 and 6 mpi (see e.g., FIG. 5C). The glioma tumor areas exhibited low proliferative indices (4-6% Ki67⁺ cells), within the upper range of proliferation rates seen in pediatric PAs, whereas the embryonal-like areas exhibited slightly higher proliferative rates (8-15% Ki67⁺ cells) (see e.g., FIG. 5A and FIG. 5C). There was no evidence of increased apoptosis (0.38-0.41% TUNEL⁺ cells; see e.g., FIG. 5D) or increased cellular senescence (0.3-0.4% β-galactosidase⁺ cells; see e.g., FIG. 6) in vivo. Taken together, these findings demonstrate that iNPCs generate both NF1-associated and sporadic LGGs in vivo.

hiPSC-Derived Glial Restricted Progenitors (iGRPs) and Oligodendrocyte Progenitors (iOPCs) form LGGs in Rag1^{-/-} Mice

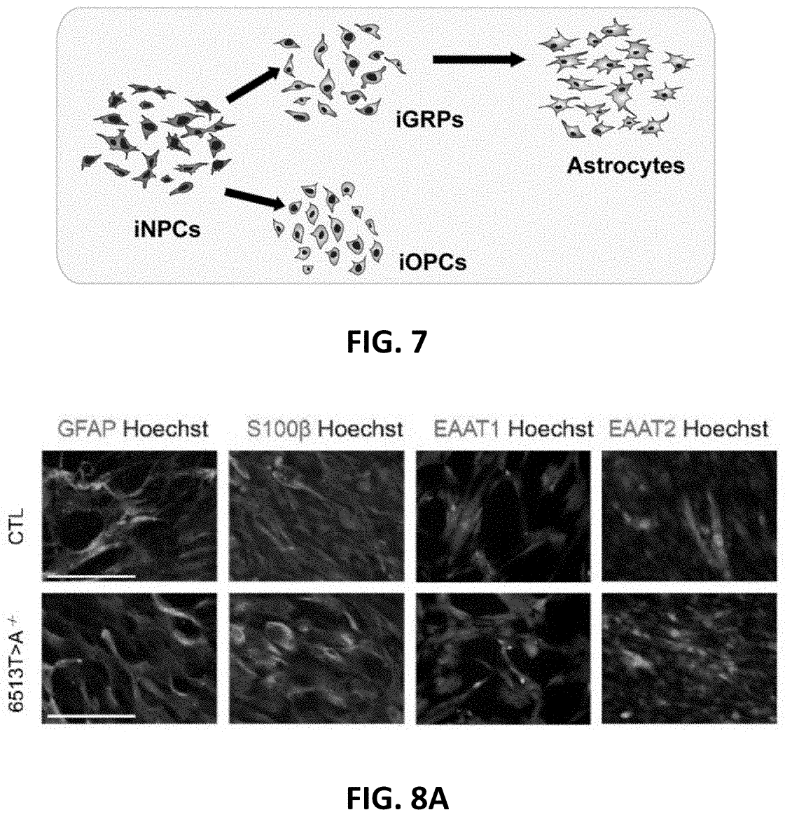

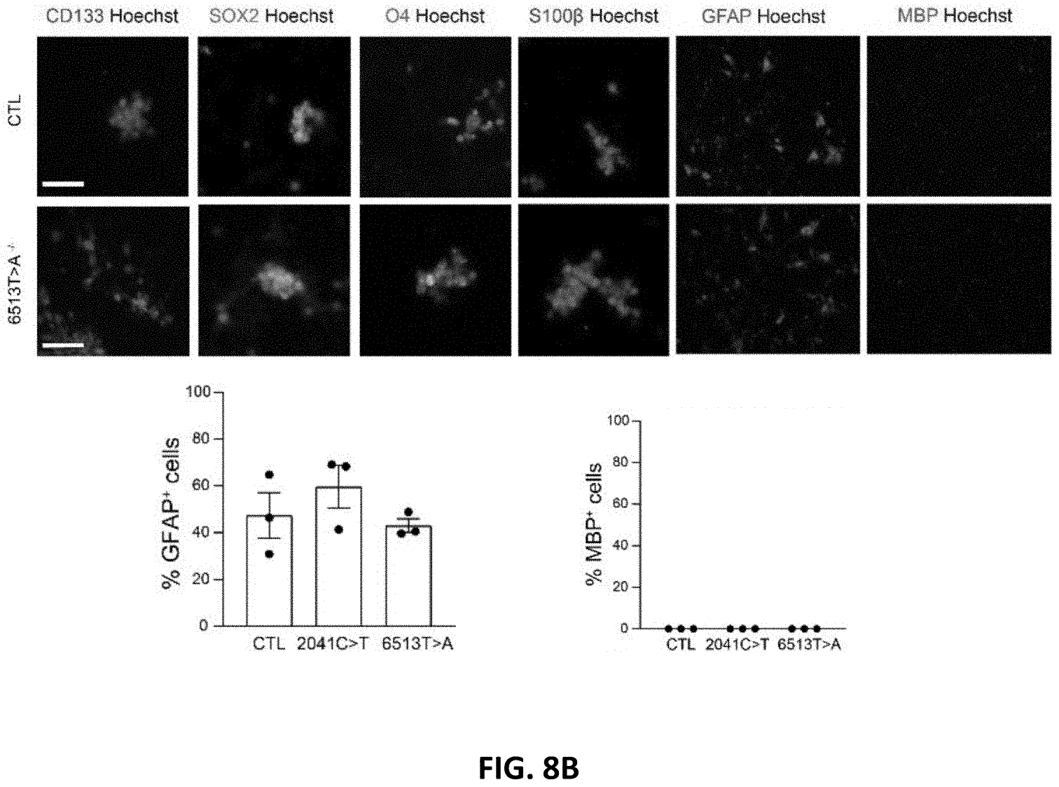

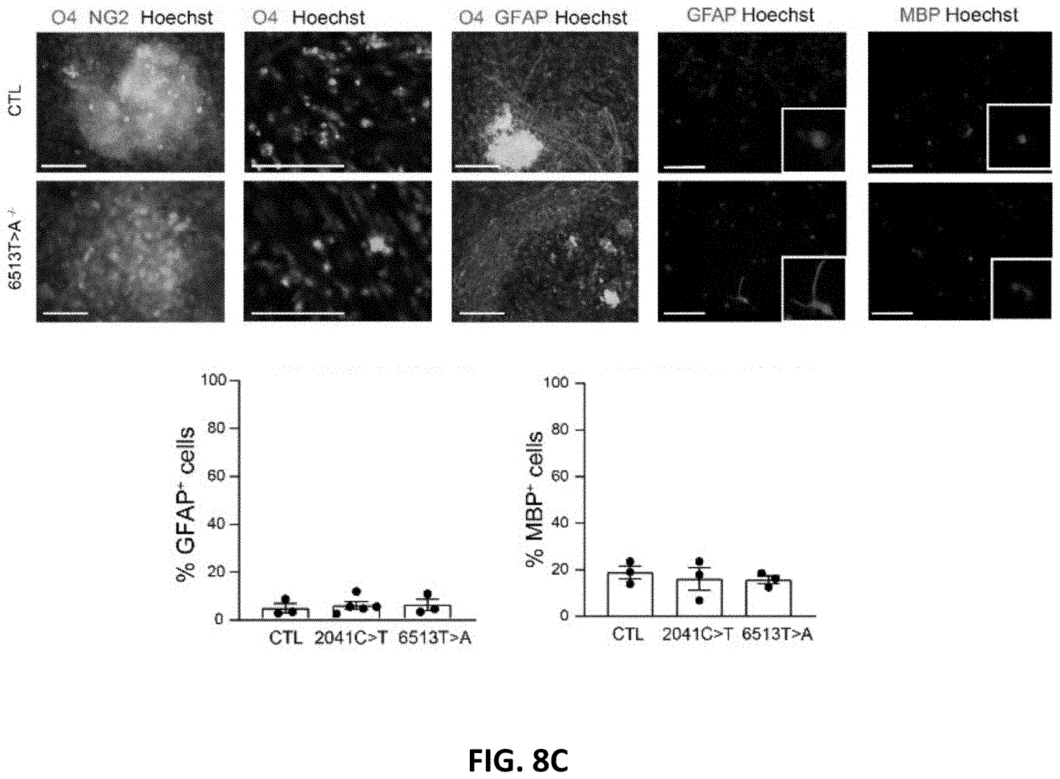

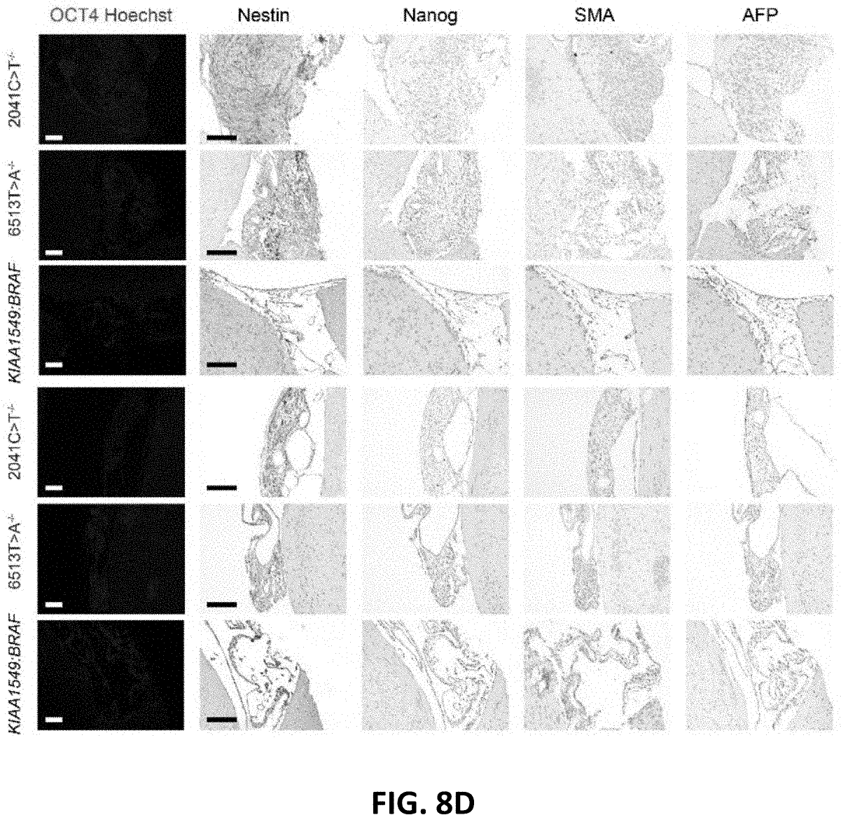

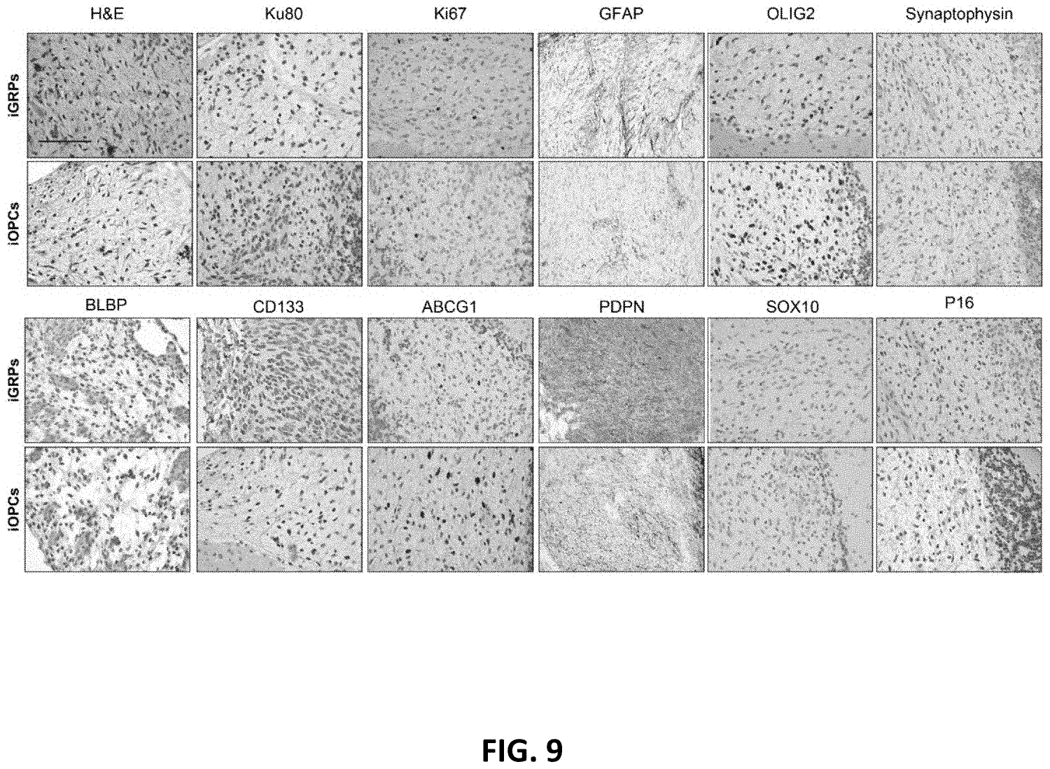

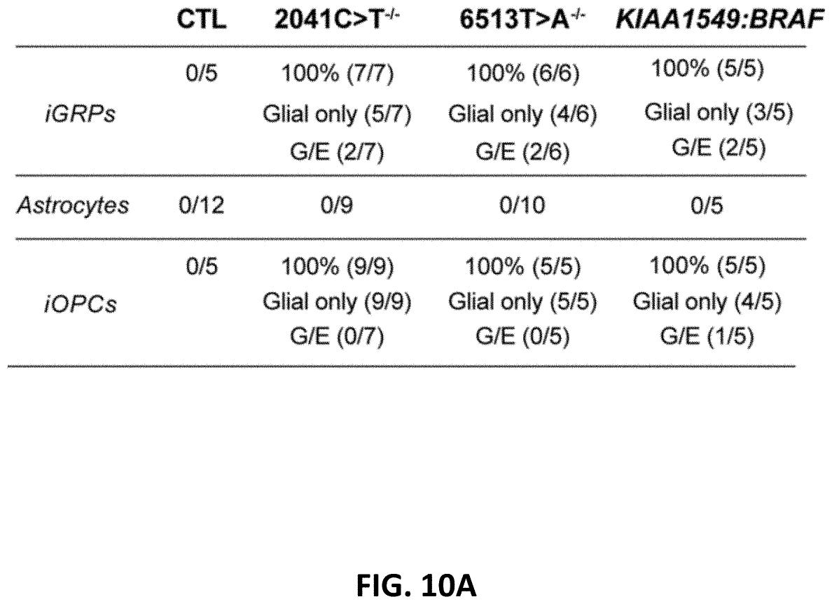

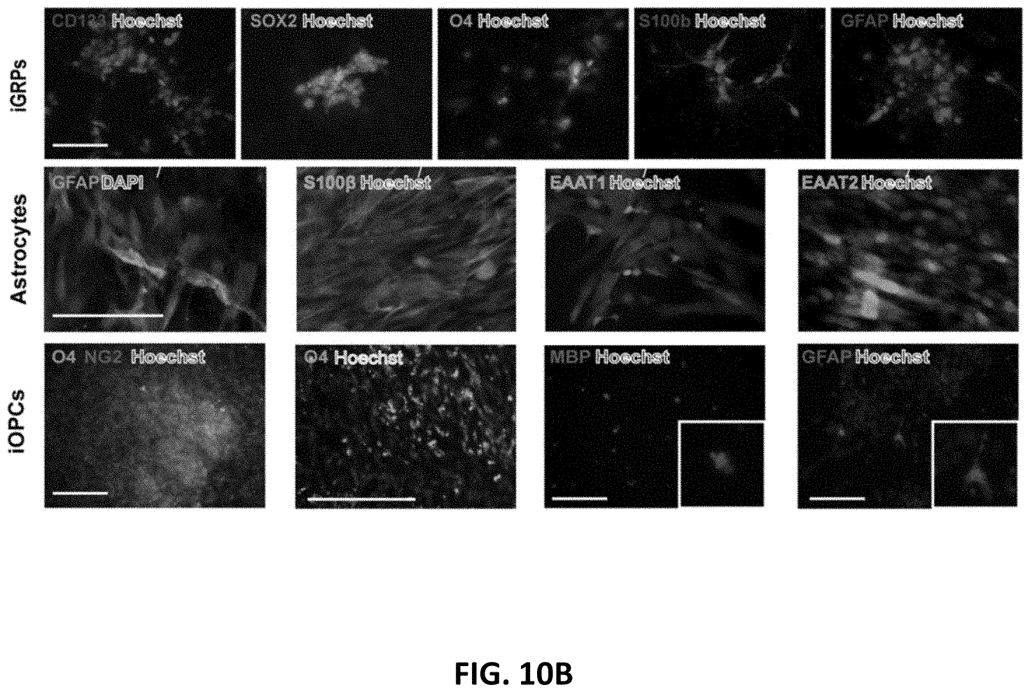

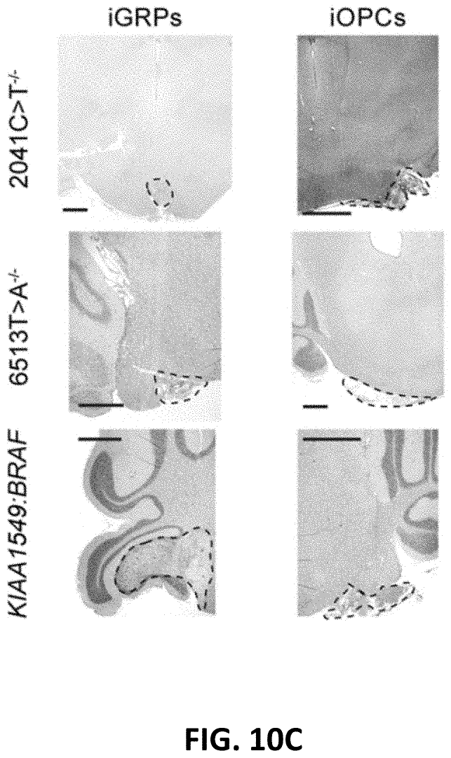

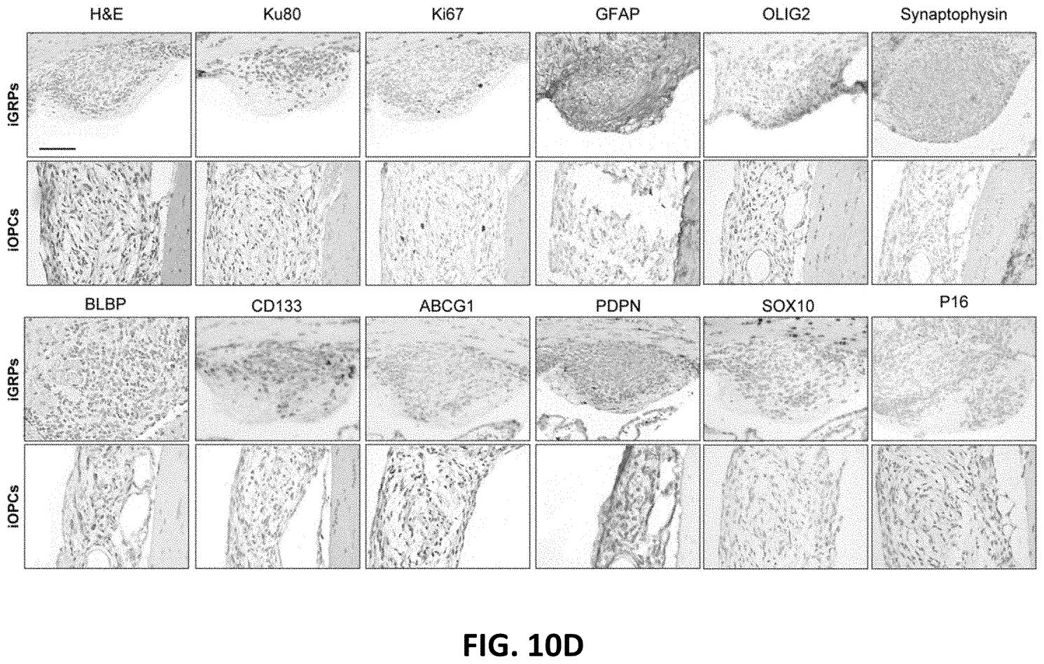

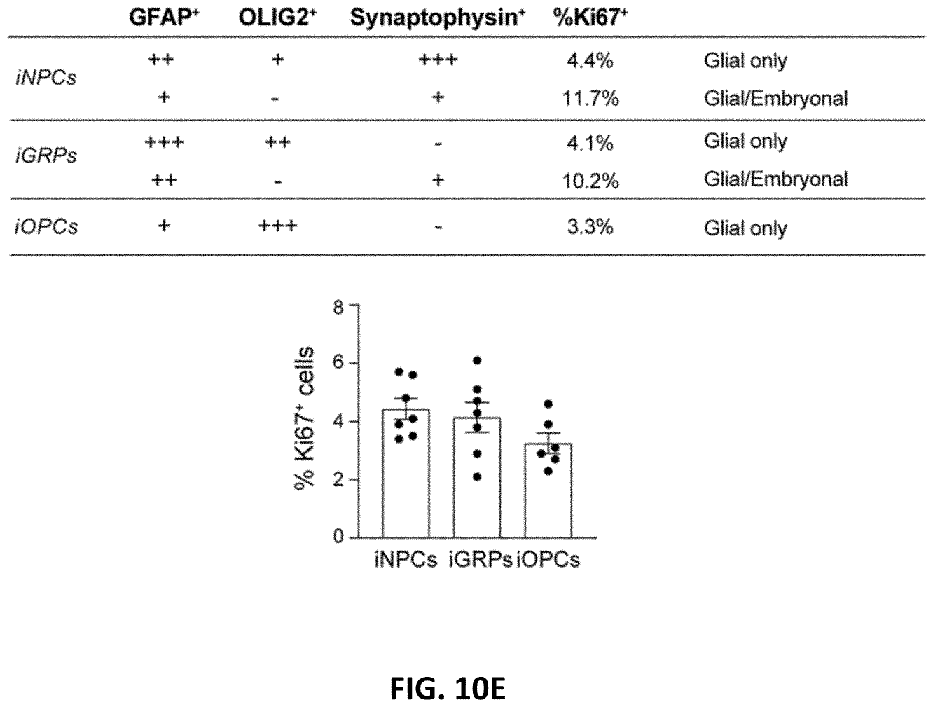

Because iNPCs are a multipotent population that generates neuronal, glial and oligodendroglial progenitor-like cells in vitro and within the LGGs in vivo (see e.g., FIG. 3D), this hiPSC platform was leveraged to examine the potential of derivative restricted progenitors to serve as cells of origin for LGGs. To this end, control, NF1-null, and KIAA1549:BRAF-expressing iNPCs were differentiated into glial restricted progenitors (iGRPs: CD133⁺, SOX2⁺, O4⁺, GFAP⁺ and S100β⁺ cells), oligodendrocyte progenitor cells (iOPCs: O4⁺, MBP⁺, GFAP⁺ and NG2^{neg} cells), and astrocytes (GFAP⁺, S100β⁺, EAAT1⁺ and EAAT2⁺ cells) (see e.g., FIG. 7) for injection into the brainstems of Rag1^{-/-} mice (see e.g., FIG. 8A-FIG. 8D and FIG. 9). 1 month following the injection of 5×10⁵ NF1-null or KIAA1549:BRAF-expressing cells, transplanted astrocytes did not form tumors (see e.g., FIG. 10A), suggesting that terminally-differentiated preneoplastic astrocytes are unlikely to be the cells of origin for pediatric LGGs, similar to that observed for murine Nf1 optic gliomas. In contrast, LGGs formed in 100% of Rag1^{-/-} mice injected with 5×10⁵ NF1-null (n=13) and KIAA1549:BRAF-expressing (n=5) iGRPs or 5×10⁵ NF1-null (n=14) and KIAA1549:BRAF-expressing (n=5) iOPCs (see e.g., FIG. 10A, FIG. 10C, and FIG. 9).

While both iGRPs and iOPCs formed LGGs, they each exhibited unique histopathologic features seen in human PAs: iGRP LGGs were compact and hypercellular, forming as either purely parenchymal masses or parenchymal masses with exophytic components. A small subset of iGRP LGGs contained glial/embryonal tumor areas that were moderately immunopositive for synaptophysin see e.g., FIG. 100, FIG. 10D, and FIG. 9). In contrast, iOPC LGGs had a looser, less compact architecture with numerous microcysts, and were located exophytically between the midbrain/brainstem and the hippocampus. In contrast to the strongly GFAP-immunoreactive iGRP LGGs, iOPC LGGs were more intensely OLIG2-immunoreactive and mostly negative for GFAP and synaptophysin expression (see e.g., FIG. 100, FIG. 10D, and FIG. 9). Both iGRP and iOPC-LGGs exhibited low proliferative indexes (4.1% and 3.2% Ki67⁺ cells, respectively) (see e.g., FIG. 10E), similar to most childhood brainstem pediatric LGGs.

iNPC-LGG Formation Requires CD4⁺ T Cell Depletion

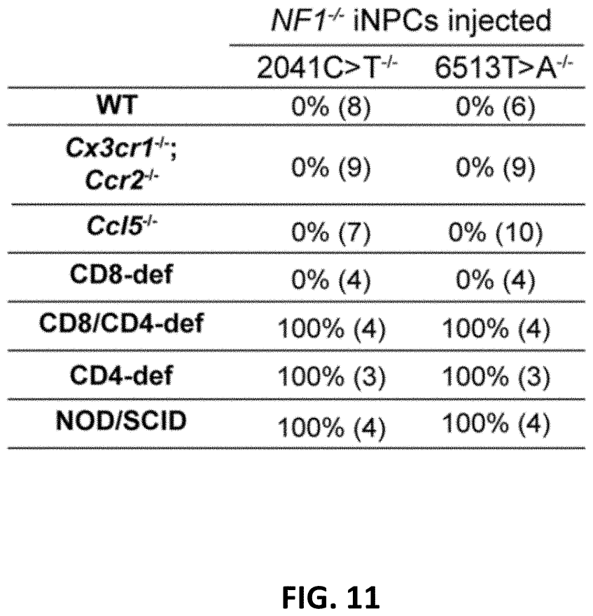

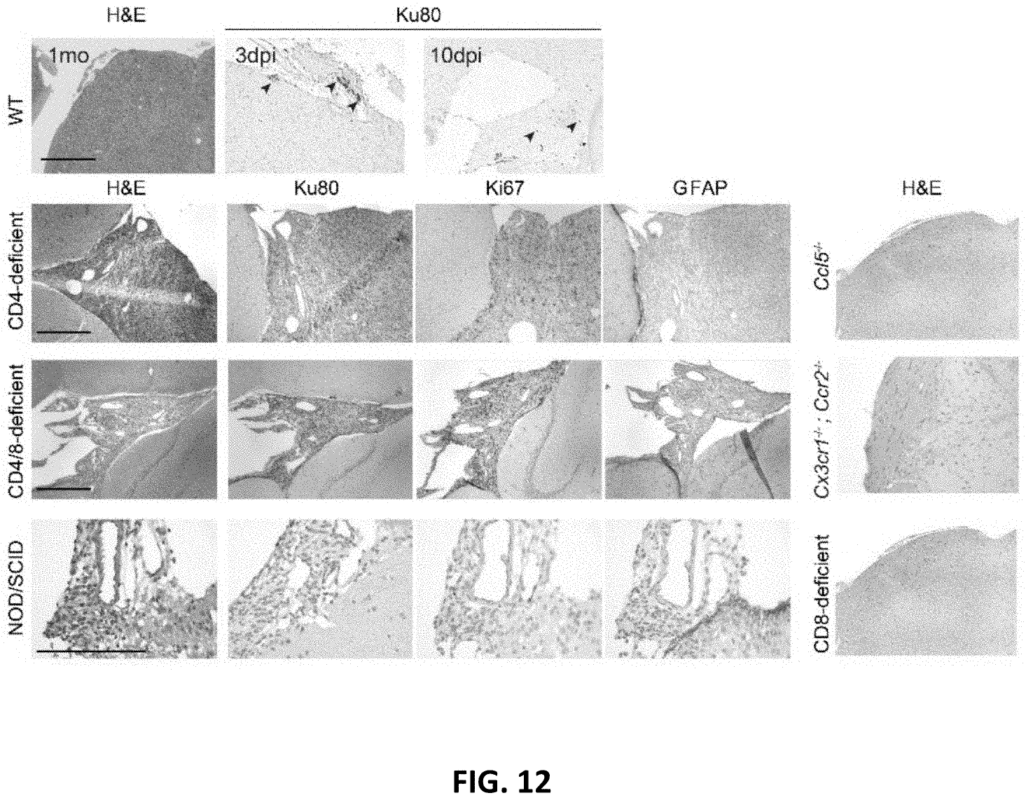

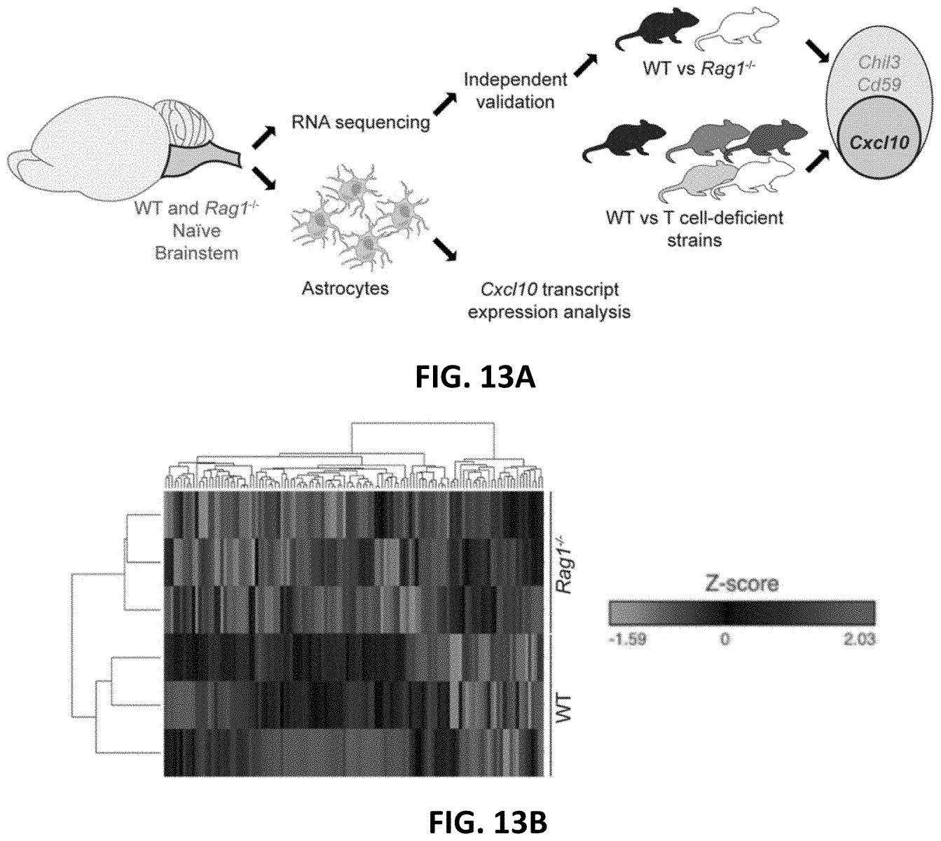

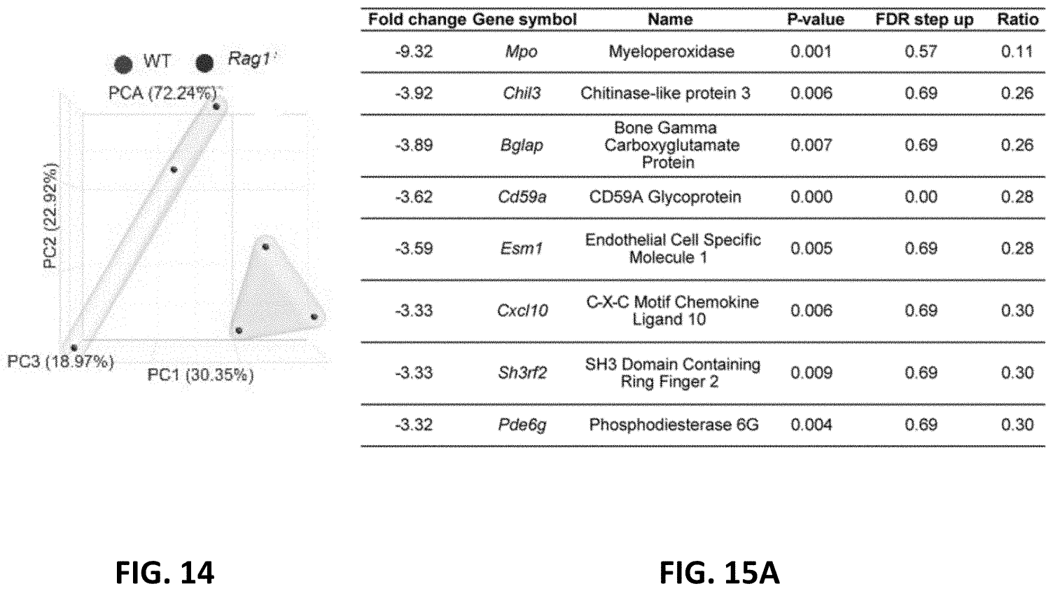

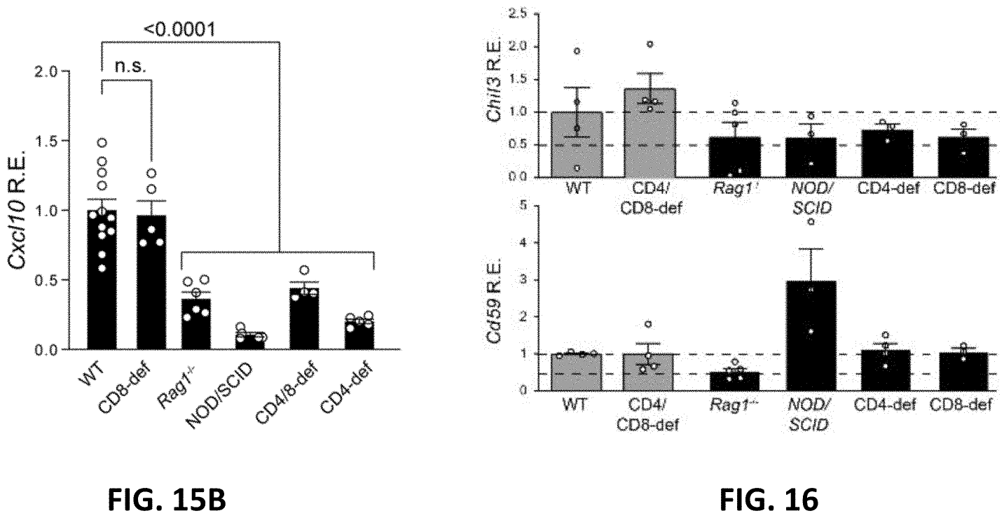

Similar to human patient brain tumor xenografts, iNPC lineage cells did not form LGGs following injection into wild type mice (see e.g., FIG. 11). To identify mouse strains that permit LGG formation, NF1-null iNPCs were injected into the brainstems of a series of immunodefective mouse strains (see e.g., FIG. 11). Whereas LGGs readily formed in NOD/SCID, CD4-deficient and CD4/CD8-deficient mice at 1 mpi, no tumors developed in CD8-deficient mice or strains deficient in the expression of microglia or T cell chemokine receptors (Cx3cr1, Ccr2), components required for murine Nf1 optic glioma formation and growth (see e.g., FIG. 12). To identify potential responsible molecular etiologies underlying CD4⁺ T cell deficiency-mediated LGG formation, transcriptomal analysis was performed on whole brainstems of wild type and Rag1^{-/-} mice (see e.g., FIG. 13A-FIG. 13B and FIG. 14). Eight downregulated transcripts and one upregulated transcript were initially identified in Rag1^{-/-} relative to wild type mouse brainstems (see e.g., FIG. 15A). While three downregulated transcripts were validated in independently-acquired Rag1^{-/-} brainstem samples (Chil3, Cd59, Cxcl10; see e.g., FIG. 15B and FIG. 16), only Cxcl10 expression was decreased in all immunodefective mouse strains that permitted LGG formation.

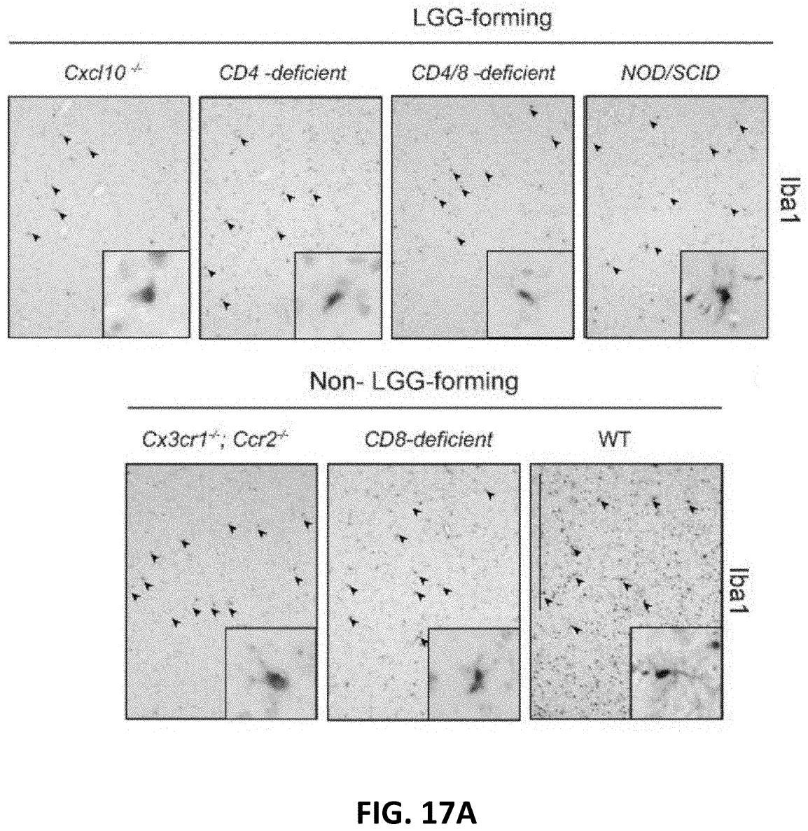

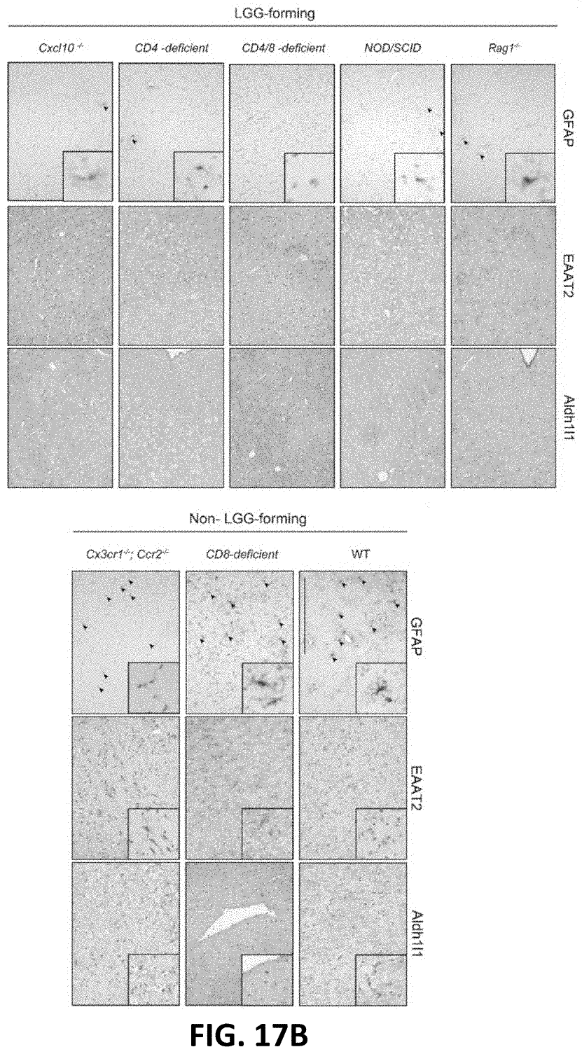

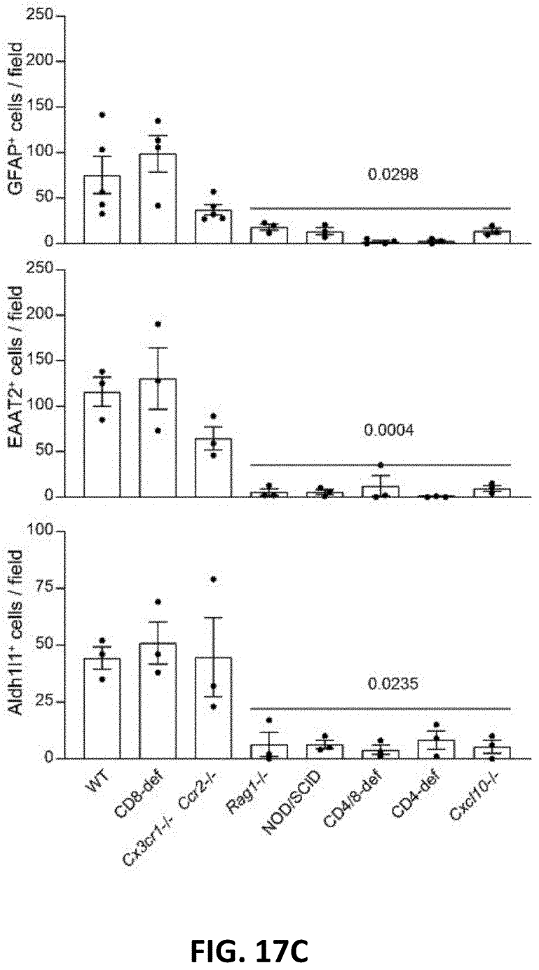

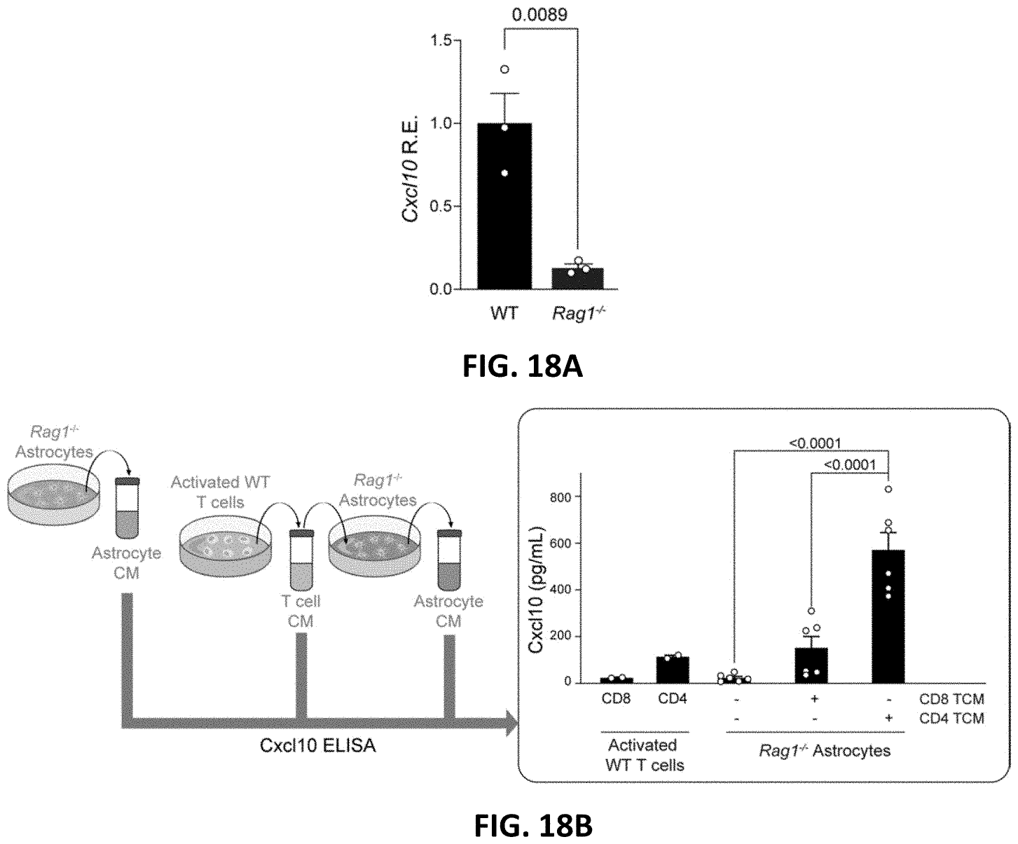

Since Cxcl10, a cytokine belonging to the CXC chemokine family, is predominantly expressed by microglia and astrocytes, these cell types were next analyzed in uninjected wild type and immunodeficient mouse brainstems. Whereas microglia density and morphology were relatively unaltered in all uninjected mouse brains analyzed (see e.g., FIG. 17A and FIG. 17C), there were fewer GFAP⁺, EAAT2⁺ and ALDH1L1⁺ astrocytes in the brainstems of all immunodefective mouse strains that permitted LGG formation (see e.g., FIG. 17B and FIG. 17C). This finding suggested that LGG formation may require a deficit in astrocytes. Consistent with an astrocyte defect, astrocytes isolated from Rag1^{-/-} mice had reduced Cxcl10 expression relative to wild type controls (86.3% reduction, see e.g., FIG. 18A). Importantly, incubation of Rag1^{-/-} astrocytes with activated wild type mouse T cell-conditioned medium (TCM) induced Cxcl10 protein production. The greatest increase in Cxcl10 protein production was observed after induction of Rag1^{-/-} astrocytes with TCM from CD4+ (24.4-fold), relative to CD8+ (6.5-fold), T cells (see e.g., FIG. 18B). Taken together, these results indicate that reduced astrocytic Cxcl10 expression in glioma-bearing mouse strains likely reflects an absence of CD4 T cells, which induce Cxcl10 expression in astrocytes to hinder LGG growth in vivo.

Cxcl10 Inhibits Pediatric LGG Formation

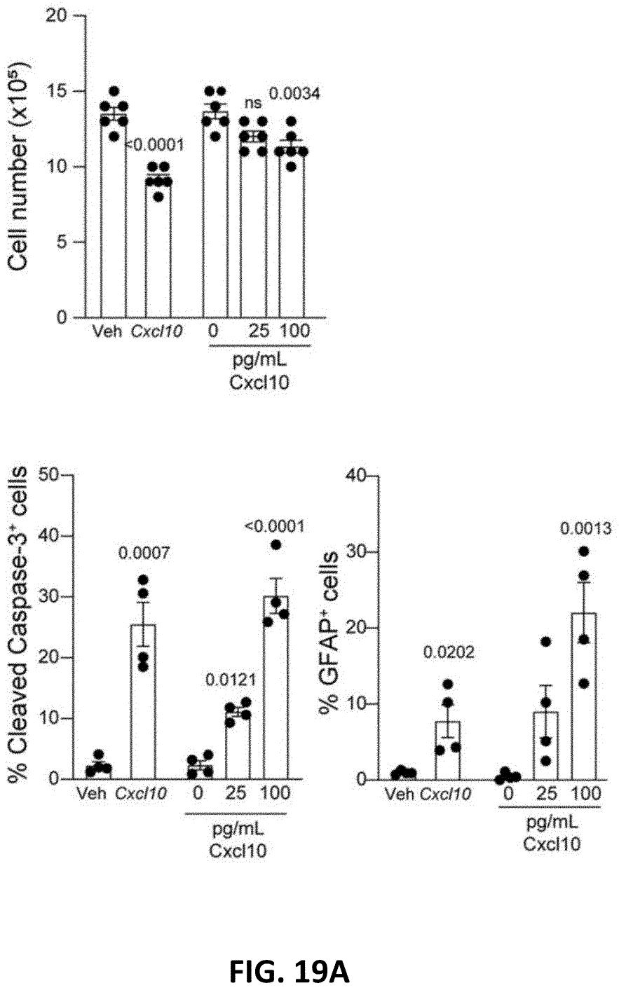

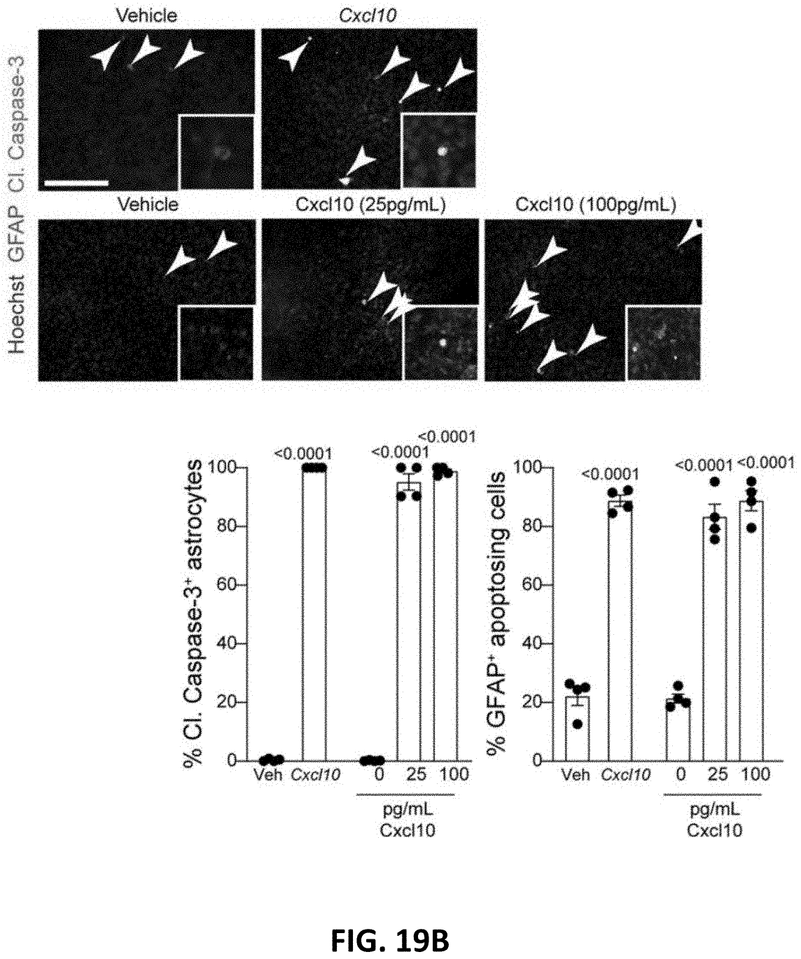

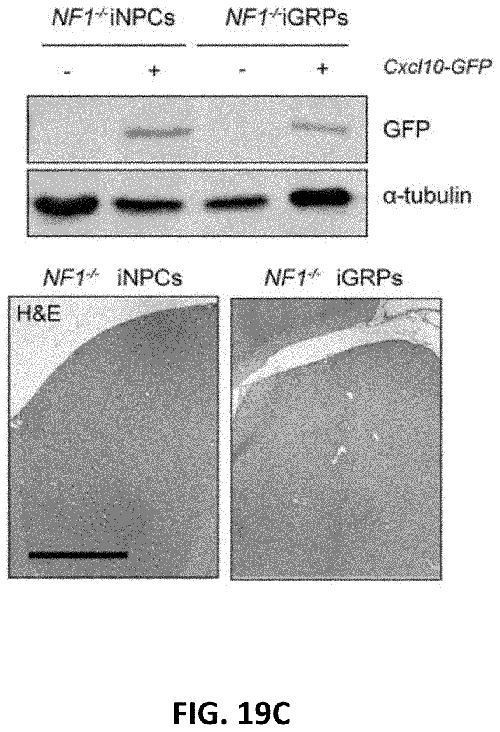

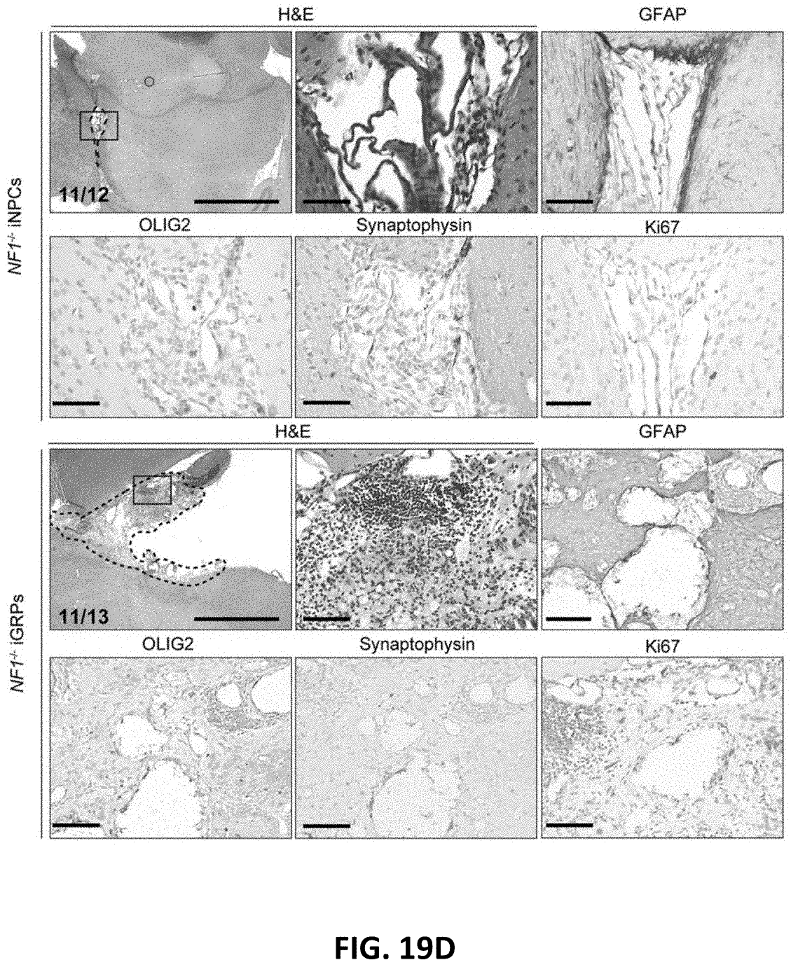

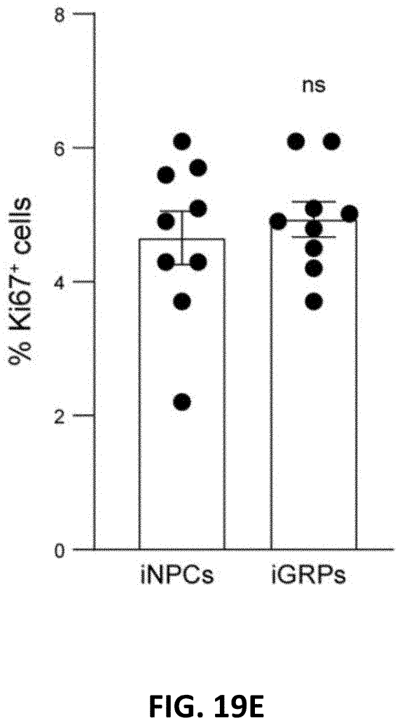

To determine whether Cxcl10 inhibits cell cycle progression or induces progenitor cell differentiation, NF1-null iNPCs were either engineered to ectopically express Cxcl10 or incubated with increasing concentrations of recombinant murine Cxcl10 protein. Both treatments induced a decrease in cell number (ectopic Cxcl10 expression, 20%; 25 pg/mL Cxcl10, 9%; 100 pg/mL Cxcl10, 13% decrease; see e.g., FIG. 19A) and an increase in programmed cell death (cleaved caspase-3+ cells; ectopic Cxcl10 expression, 9.4-fold; 25 pg/mL Cxcl10, 4.6-fold; 100 pg/mL Cxcl10, 20.5-fold increase; see e.g., FIG. 19B). Additionally, both treatments increased GFAP⁺ astrocytic differentiation of the pluripotent iNPCs (ectopic Cxcl10 expression, 8.3-fold; 25 pg/mL Cxcl10, 8.2-fold; 100 pg/mL Cxcl10, 20.5-fold increase; see e.g., FIG. 19B). In this respect, 95-100% of the differentiated GFAP⁺ cells were cleaved caspase-3-positive, and 83.3-88.8% of the total number of cells undergoing apoptosis were astrocytes (see e.g., FIG. 19B). Together, these results demonstrate that Cxcl10 induces astrocytic differentiation, as well as cell death, in vitro.

To demonstrate that stromal Cxcl10 is sufficient to inhibit LGG formation in vivo, NF1-null iNPCs and iGRPs engineered to ectopically express murine Cxcl10 were injected into the brainstems of Rag1^{-/-} mice. In contrast to vector-infected controls, no LGGs formed in mice following the injection of NF1-null iNPCs and iGRPs expressing murine Cxcl10 (see e.g., FIG. 19C). Conversely, to establish that Cxcl10 is necessary to inhibit LGG formation, NF1-null iNPCs and iGRPs were transplanted into the brainstems of Cxcl10^{-/-} mice. At 1 mpi, 92% and 85% of the injected mice developed iNPC- and iGRP-derived LGGs, respectively (see e.g., FIG. 19D). These lesions were hypercellular, exophytic, and immunopositive for GFAP and OLIG2 expression, with 4.9% and 4.9% Ki67⁺ cells, respectively (see e.g., FIG. 19E). Taken together, these data demonstrate that Cxcl10 is both necessary and sufficient to suppress LGG cell growth both in vitro and in vivo.

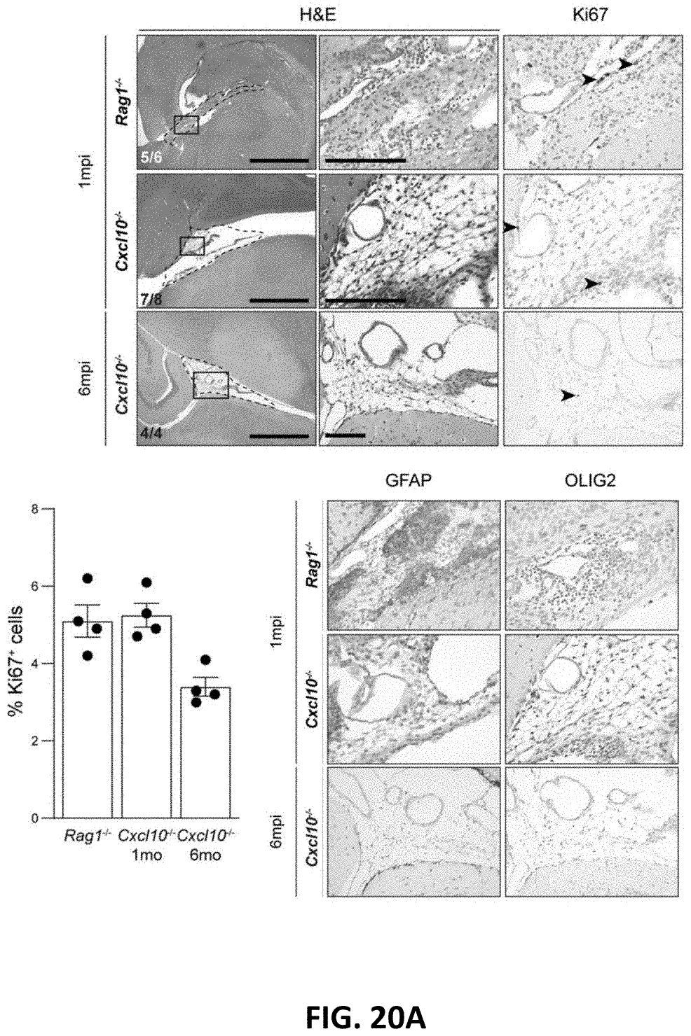

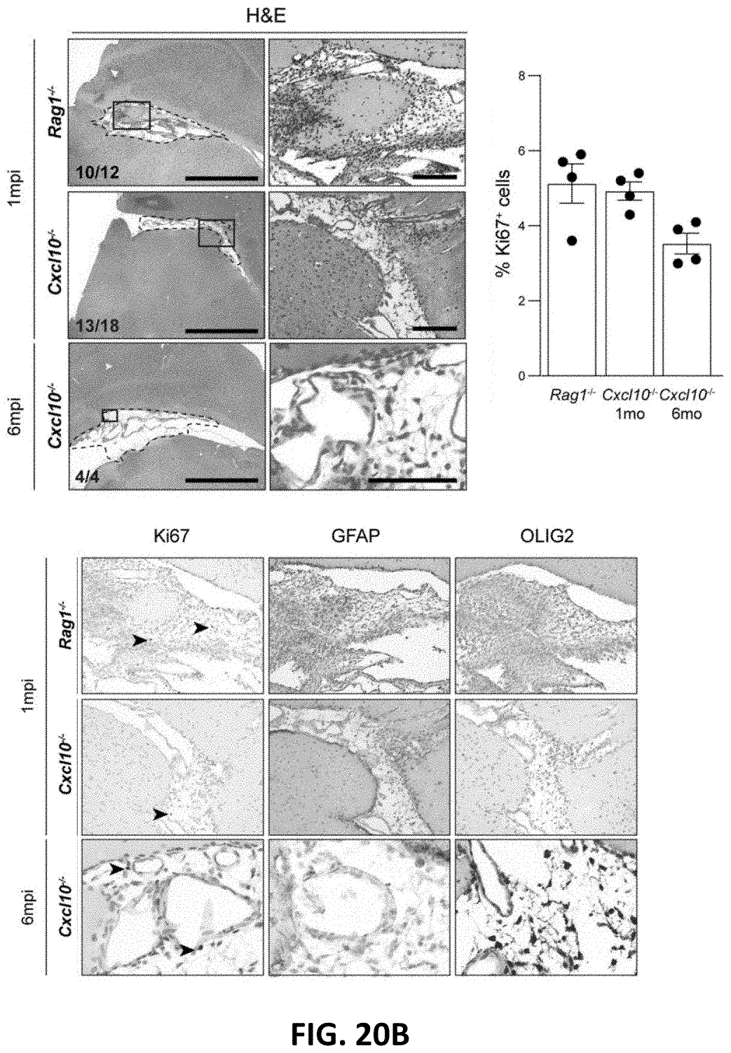

Human pediatric LGG cell lines develop LGGs in Cxcl10^{-/-} mice To extend these findings to human PDX modeling, two primary human PA cell lines from one patient with an NF1-PA (JHH-NF-PA; NF1 loss) and another with a sporadic PA (Res186) were leveraged. While these lines grow briefly in larval zebrafish (6 days), they do not form tumors in athymic nude mice over the course of 12 months. Similar to the experiments using hiPSC neuroglial lineage cells, 5×10⁵ PA cell lines were injected into brainstems of Rag1^{-/-} and Cxcl10^{-/-} mice. Whereas human pediatric PA cells did not form gliomas in wild type mice, both Rag1^{-/-} and Cxcl10^{-/-} mice developed LGGs at 1 mpi and 6 mpi. These LGGs were hypercellular with microcystic components, parenchymal with exophytic components, immunopositive for OLIG2, but mostly negative for GFAP, expression (see e.g., FIG. 20A-FIG. 20B). These lesions exhibited proliferative indices between 4.2 and 4.7% at 1 mpi and 3.4-3.5% at 6 mpi. Together, these results establish Cxcl10^{-/-} mice as a tractable in vivo platform for human pediatric LGG modeling.

hiPSC-LGG Growth is Reduced Following MEK Inhibition

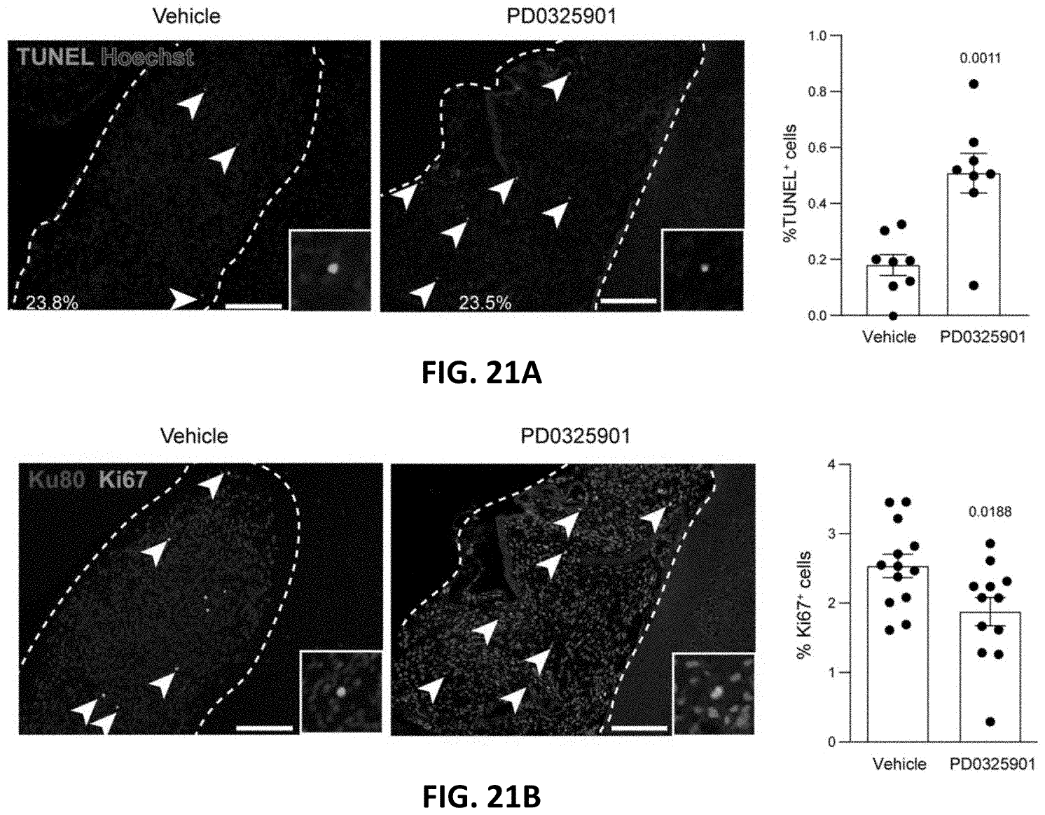

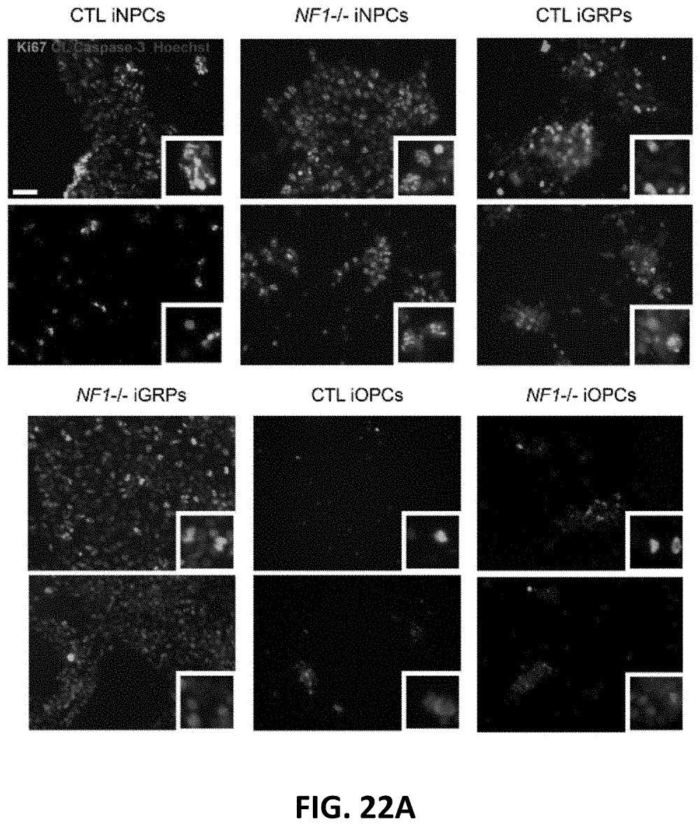

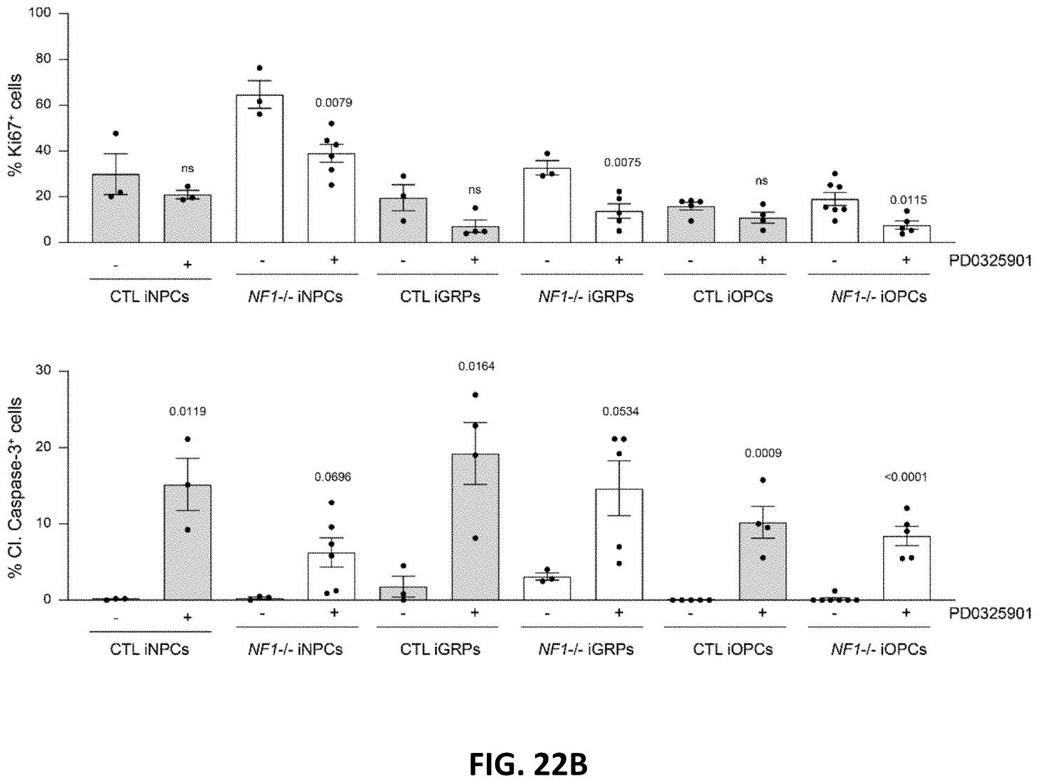

Finally, to provide a proof-of-principle demonstration that this humanized LGG platform could be used for preclinical drug evaluation, the ability of a MEK inhibitor (PD0325901) to block hiPSC-LGG growth was assayed beginning at 1 mpi in vivo. The treatment lasted a total of four weeks, a time frame previously reported to inhibit Nf1 mouse low-grade optic glioma growth. While Ku80⁺ iNPC-LGGs were still detectable in mice following PD0325901 administration, there was increased tumor cell apoptosis (TUNEL⁺ cells), as well as reduced tumor cell proliferation (Ki67⁺ cells), relative to vehicle-treated LGGs (see e.g., FIG. 21A-FIG. 21B). In vitro PD0325901 treatment of NF1 null iNPCs, iGRPs and iOPCs also resulted in decreased cell proliferation and increased apoptosis (see e.g., FIG. 22A-FIG. 22B), thus establishing this experimental platform for preclinical therapeutic studies.

Discussion

Building upon prior studies using hiPSC-derived 2D and 3D organoid cultures to study high-grade gliomas and medulloblastoma, a humanized xenograft platform was developed herein to model sporadic and NF1-associated pediatric LGGs and elucidate the pathogenesis, cellular origins, and signaling pathway dependencies. Beyond the value of this system to PDX pediatric LGG future preclinical experimentation, this study raises several important points germane to human brain tumor pathobiology.

First, leveraging hiPSC engineering for humanized tumor modeling represents an efficient system applicable to other low-grade nervous system tumor xenografts, which have been extremely challenging to establish in mice. Relevant to pediatric LGGs, like NF1- PA and BRAF-driven PAs, the derivative tumor cells undergo oncogene-induced senescence and display a senescence-associated secretory phenotype (SASP) in vitro unless provided with fibroblast conditioned medium and ROCK inhibition or senolytic inhibitors. The fragility of these PA tumor cell cultures likely reflects their profound stromal (tumor microenvironment) dependence, as well as their limited intrinsic self-renewal capacity, as demonstrated using genetically engineered mouse models and murine pediatric LGG explant systems. In these studies, Nf1 optic glioma growth in mice requires T cell and microglia support through the elaboration of critical cytokines and growth factors, as Nf1 optic glioma stem cells cannot form glioma-like lesions in mice lacking these stromal cells. Similarly, KIAA1549:BRAF-expressing cerebellar stem cells cannot form tumors in mice lacking the T cell and microglia Ccr2 chemokine receptor. Moreover, the SASP represents a cellular state characterized by the secretion of inflammatory cytokines and immune modulators, which may counter the pro-tumoral support provided by T cells and monocytes in the tumor microenvironment.

Second, the hiPSC-LGG explant system provides a tractable platform to define the putative cells of origin for histologically distinct tumors, as well as histologically similar tumors arising in different locations. This is particularly important with respect to the cellular ontogeny of brain cancers. In this regard, previous mouse modeling experiments have demonstrated that multiple cell types, including differentiated cells (astrocytes, neurons), can give rise to high-grade gliomas. Using other murine modeling approaches, NPCs, or their derivative progenitor cells, have been shown to be the putative cells of origin, while restricted progenitors of the NPC lineage give rise to oligodendrogliomas and proneural glioblastomas. Moreover, additional cellular constraints, including the specific neuroglial progenitor population and the particular brain location (third ventricle versus lateral ventricle germinal zone) are critical determinants that dictate glioma formation and latency. In this report, specific human neuroglial lineage cells were leveraged to determine that not all lineages faithfully recapitulate LGG histological features. Whereas astrocytes do not give rise to these tumors, iGRPs, iOPCs and iNPCs with NF1 loss or KIAA1549:BRAF expression form LGGs within a month. It is intriguing to note that iGRPs give rise to more compact tumors with stronger GFAP than OLIG2 immunoreactivity, resembling optic pathway and brainstem gliomas, while the iOPC-derived lesions have looser stroma with microcystic changes and a higher density of OLIG2⁺ than GFAP⁺ cells, similar to many cerebellar human PAs. In contrast, Olig2⁺, NG2^{neg} glial progenitors form Nf1 optic gliomas with delayed latency, which may reflect innate differences between neuroglial cell populations in humans and rodents and/or different potential cells of origin.

Third, the finding that humanized pediatric LGGs develop in a mouse strain not lacking immune cells opens a new avenue for the in vivo study of low-grade PDXs. Whereas some orthotopically injected high-grade PDXs grow in wild type mice, the majority of PDX studies leverage immunocompromised (usually athymic and NOD/SCID) mice, due to their inherent inability to reject engrafted human cells. However, even in these immune-impaired animals, not all tumors are able to grow, possibly due to the lack of a trophic environment provided by a complete immune system. Since T cells present in human PAs and Nf1 murine low-grade gliomas establish a supportive microenvironment for low-grade glioma growth in vivo, it is important to use host systems with immune systems with limited immunologic impairment, such as Cxcl10^{-/-} mice. Additional studies are in progress to define immune function in Cxcl10-deficient mice. The relevance of Cxcl10 production to xenograft establishment and tumorigenesis is further strengthened by prior reports demonstrating that elevated CXCL10 in bronchoalveolar lavage fluid is associated with acute lung transplant rejection and that viral CXCL10 gene therapy improves cervical cancer xenograft responses to radiotherapy. In addition, increased Cxcl10 expression is inversely correlated with tumor growth and is associated with cardiac allograft rejections in the PNS, such that enhanced systemic Cxcl10 expression is a prognostic biomarker of graft-versus-host disease. Moreover, reduced Cxcl10 levels in HGG spheroids caused by long-term in vitro culture correlates with higher engraftment rate in immunocompetent rats. Finally, CXCL10 is induced as part of the SASP, which limits primary human LGG cell growth in vitro through increased senescence. Understanding the mechanisms underlying CXCL10-mediated hiPSC and patient-derived xenograft growth inhibition will be critical to the development of second-generation PDX models of pediatric LGGs.

Conclusions

Rag1^{-/-} mice have impaired immune systems and the resulting LGGs lack some of the non-neoplastic cell types (e.g., T cells) important for murine LGG growth. Similarly, the impact of Cxcl10 loss on immune system function is unknown. Ongoing studies are focused on co-introducing hiPSC-derived monocyte populations, as well as defining the impact of Cxcl10 loss on non-neoplastic cell function. While future studies may create preclinical models that fully recapitulate all of the elements of pediatric LGGs, the model generated herein is an experimentally tractable in vivo platform for humanized orthotopic pediatric LGG modeling. This model has great potential to galvanize the study of a variety of different types of brain and nerve tumors, including previously unexplored low-grade subtypes, as well as advance understanding of the molecular and cellular origins of these common brain tumors in children.

Materials and Methods

Study Approval

All experiments were performed under active and approved Animal Studies Committee protocols at Washington University.

Animals

Mice were maintained on a 12 light/dark cycle in a barrier facility, had ad libitum access to food and water, were exclusively used for the purposes of this study, and had not undergone any additional procedures. Mating cages housed one male and two females of the same genotype, and males were separated from dams at the time of delivery. Injections were performed on entire litters of 0-3-day-old mice of the following strains: C57BL/6J, Rag1^{-/-} (B6.12957-Rag1tm1Mom/J; strain 002216, Jackson laboratories), Cxcl10^{-/-} (B6.12954-Cxcl10tm1Adl/J; strain 006087, Jackson laboratories), CD4-deficient (B6.12952-Cd4tm1Mak/J; strain 002663, Jackson laboratories), CD8-deficient (B6.12952-Cd8atm1Mak/J; strain 002665, Jackson laboratories), CD4/CD8-deficient (intercrossed strains 002663 and 002665), NODISCID (NOD. Cg-Prkdcscid/J; strain 001,303, Jackson laboratories), Ccl5^{-/-} (B6.129P2-Ccl5tm1Hso/J; strain 005090; Jackson laboratories) and Cx3cr1^{-/-}, Ccr2^{-/-} mice. Injected pups were allowed to recover and were subsequently immediately returned to their maternal cage until weaning. Mice of both sexes were randomly assigned to all experimental groups without bias, and the investigators were blinded until final data analysis during all of the experiments.

Human Induced Pluripotent Stem Cell Culture and Differentiation

NF1 patient homozygous and heterozygous germline NF1 gene (Transcript ID NM_000267) mutations (c.2041C>T; c.6513T>A) were CRISPR/Cas9-engineered into a single commercially available male control human iPSC line (BJFF.6) by the Washington University Genome Engineering and iPSC Core Facility (GEiC). Homozygous mutations were confirmed by NGS sequencing, and two different clones were expanded for each of the NF1^{-/-} and control lines for all subsequent differentiation procedures. Human induced pluripotent stem cells (hiPSCs) were grown on Matrigel (Corning)-coated culture flasks, and were fed daily with mTeSR Plus medium (STEMCELL Technologies). hiPSCs were passaged as needed with ReLeSR medium (STEMCELL technologies) following the manufacturer's instructions. For neural progenitor cell (iNPC) differentiation, hiPSCs were transferred to poly-1-ornithine (Sigma-Aldrich)/Laminin (Fisher)-coated tissue culture flasks and incubated for 3 days in NPC basic media [50% DMEM/F12, 50% Neurobasal medium, 1×N-2 supplement, 1×B-27 supplement, 2 mM GlutaMax (all Thermo Fisher Scientific)] supplemented with 10 ng/mL human LIF, 4 μM CHIR99021, 3 μM SB431542, 2 μM Dorsomorphin and 0.1 μM Compound E (all STEMCELL Technologies). Subsequently, cells were incubated for 5 days in NPC basic medium supplemented with 10 ng/mL human LIF, 4 μM CHIR99021, 3 μM SB431542, and 0.1 μM Compound E. Finally, iNPCs were incubated and maintained in NPC basic medium supplemented with 10 ng/mL human LIF, 3 μM CHIR99021 and 2 μM SB431542. The medium was refreshed daily, and iNPCs passaged as needed with Accutase (STEMCELL Technologies) following the manufacturer's instructions. For astrocyte differentiation, iNPCs were transferred onto Primaria-coated plates and maintained in Astrocyte growth medium (ThermoFisher Scientific). Astrocytes were fed 3 times a week and passaged as needed with 0.05% Trypsin(Fisher) following the manufacturer's instructions. For glial restricted progenitor (iGRP) differentiation, iNPCs were dissociated with Accutase (STEMCELL Technologies) following the manufacturer's instructions, and floating cells transferred to low-attachment culture flasks to allow for gliosphere formation. iGRPs were incubated for 2 weeks in the following medium: Basal GRP medium [DMEM/F12 supplemented with Sodium bicarbonate (Sigma-Aldrich), 1×B-27 Supplement (Thermo Fisher Scientific), 1×N-2 Supplement (Thermo Fisher Scientific), 1% penicillin-streptomycin (Thermo Fisher), 1% L-glutamine (Thermo Fisher Scientific), and 1% nonessential amino acids (Thermo Fisher Scientific)] supplemented with 10 ng/mL NT-3 (Peprotech), 10 μM forskolin (Tocris), 60 ng/mL 3,3′,5-triiodo-l-thyronine (T3; Sigma-Aldrich), 20 μg/mL ascorbic acid (Sigma-Aldrich) and 25 μg/mL insulin (Sigma-Aldrich). The supernatant was refreshed 3 times a week by gentle aspiration. For subsequent glial differentiation, gliospheres were incubated for 3 weeks in basal GRP medium supplemented with 20 ng/mL PDGF-AA (Peprotech), 10 ng/ mL IGF-1 (Fisher), 10 ng/mL NT-3 (Peprotech), 10 μM forskolin (Tocris), 60 ng/mL T3 (Sigma-Aldrich), and 10 μg/mL insulin (Sigma-Aldrich). The supernatant was refreshed 3 times a week by gentle aspiration. For oligodendrocyte progenitor cell (OPC) differentiation, embryoid bodies (EBs) were generated directly from iPSCs by seeding 60,000 iPSCs at the bottoms of ultralow cell attachment U-bottom 96 well plates, and incubating them for 5 days in NIM (DMEM/F12, 1% NEAA, 1×N-2 supplement). Subsequently, the EBs were transferred onto poly-l-ornithine/Laminin-coated 6-well plates and incubated for 11 days in NIM supplemented with 20 ng/mL bFGF (Peprotech) and 2 μg/mL heparin (STEMCELL technologies), 3 days in NIM supplemented with 100 nM retinoic acid (RA; Sigma-Aldrich), 7 days in NIM supplemented with 100 nM RA, 1 μM Purmorphamine (Pur; STEMCELL Technologies) and 1×B-27, and then 11 days incubation in NIM supplemented with 10 ng/mL bFGF, 1 μM Pur and 1×B-27. For OPC specification and maturation, the spheres were then transferred to low-attachment culture flasks and incubated for 120 days in glial induction medium [DMEM/F12, 1×N1 (Sigma-Aldrich), 1×B27, 60 ng/mL T3, 100 ng/mL Biotin (Sigma-Aldrich) and 1 μM cAMP (Peprotech)] supplemented with 10 ng/mL PDGF-AA, 10 ng/mL IGF-1 and 10 ng/mL NT3.

Intracranial Injections

Postnatal day 0-4 animals were anesthetized and intracranially injected in accordance with active Animal Studies Committee protocols at Washington University. 1×10⁴, 5×10⁴, 1×10⁵, or 5×10⁵ cells resuspended in 2 μL ice-cold PBS were injected 0.7 mm to the right of the midline into either the midbrain (0.5 mm posterior to Lambda; 2 mm deep), or the cerebellum (2 mm posterior to Lambda; 1 mm deep) of neonatal mice using a Hamilton syringe. Animals were aged to 1, 3 or 6 months post injection prior to tissue harvesting and analysis.

PD0325901 Treatments

Twenty 4-week-old Rag1^{-/-} mice of both sexes harboring NF1-null iNPC-LGGs 1 month post-injection were intraperitoneally injected either with 10% DMSO in saline or with 5 mg/kg/day PD0325901 (Sigma-Aldrich), 6 days a week, for a total of four weeks. Treated mice were collected for histopathologic analysis. For in vitro experiments, iNPCs, iGRPs and iOPCs were treated with 10 nM PD0325901 for 24 h prior to immunocytochemical analysis.

Magnetic Resonance Imaging (MRI)

Injected mice were transcardially perfused with Ringer's solution and 4% paraformaldehyde (PFA). The entire brain was removed from the animals and post-fixed in 4% PFA overnight prior to rehydration in phosphate buffered saline (PBS) for a minimum of 7 days. Rehydrated brain samples underwent MRI. Each mouse brain was then packed into a 2 mL plastic vial, supported with fiberglass, and immersed in Fluorinert (FC-3283; 3 M Company, St. Paul, Minn.). MRI experiments were performed using a 4.7-T small-animal MR scanner built around an AgilentNarian (Santa Clara, Calif.) DirectDrive™ console and an Oxford Instruments (Oxford, United Kingdom) horizontal-bore superconducting magnet. The plastic vial containing the mouse brain was loaded into a laboratory built solenoid RF coil (1-cm diameter; 2-cm length). MR images were collected with a 3D gradient echo (GE3D) sequence: TR 5.0 ms, TE 2.2 ms, flip angle 30°, matrix size 128×128×128, FOV 16×16×16 mm³, 0.19 mm isotropic resolution, 4 averages, and 5.5 min data acquisition time. The images were loaded into MatLab (Math-Works®, Natick, Mass.) and converted into NIfTI (.nii) format for tumor inspection and segmentation with ITKSNAP.

Tissue Fixation and Immunohistochemistry

Injected mice were transcardially perfused at 1, 3 or 6 months post-injection, initially with Ringer's solution, and then followed by ice-cold 4% paraformaldehyde. Whole mouse brains were harvested, post-fixed in 4% PFA and dehydrated in 70% ethanol. Dehydrated samples were then paraffin-embedded and serially sectioned (5 μm). Hematoxylin and eosin (H&E), as well as antibody immunohistochemical staining, were performed using the primary antibodies, Vectastain ABC kit (Vector Laboratories) and appropriate biotinylated secondary antibodies provided in TABLE 1.

TABLE 1
Antibodies used.

| Antibody | Manufacturer | Catalog Number |
|---|---|---|
| Anti-ABCG1 antibody | GeneTex | GTX30598 |
| Anti-ALDH1L1 antibody | Abcam | ab87117 |
| Anti-Alpha-1 Fetoprotein | Abcam | ab169552 |
| (AFP) antibody | | |
| [EPR9309] | | |
| Anti-cleaved caspase-3 | Cell Signaling | 9661S |
| (Asp175) antibody | Technologies | |
| Anti-CD133 antibody | Abcam | ab19898 |
| Anti-CD28 antibody | Fisher Scientific | 16-0281-82 |
| Anti-CDKN2A/p16INK4a | Abcam | ab54210 |
| antibody [2D9A12] | | |
| Anti-EAAT1 antibody | Abcam | ab416 |
| Anti-EAAT2 antibody | Abcam | ab203130 |
| Anti-GFAP/Glial | Fisher | 13-0300 |
| Fibrillary Acid Protein | | |
| antibody (2.2B10) | | |
| Anti-Ki67, Clone B56 | BD Biosciences | BDB556003 |
| Anti-Ku80 (C48E7) | Cell Signaling | 2180S |
| antibody | | |
| Anti-MBP antibody | Abcam | ab62631 |
| Anti-Nestin antibody | Abcam | ab92391 |
| (ICC) | | |
| Anti-Nestin antibody | Abcam | ab18102 |
| (IHC) | | |
| Anti-NG2 antibody | Abcam | ab129051 |
| Anti-O4 antibody, clone | Fisher Scientific | MAB345MI |
| 81 | | |
| Anti-OLIG2 antibody | GeneTex | GTX132732 |
| Anti-p44/42 MAPK | Cell Signaling | 9102S |
| (Erk1/2) antibody | Technologies | |
| Anti-Phospho-p44/42 | Cell Signaling | 9101S |
| MAPK (Erk1/2) | Technologies | |
| (Thr202/Thr204) | | |
| antibody | | |
| Anti-PDPN antibody | Abcam | ab236529 |
| Anti-S100β antibody | Abcam | ab52642 |
| Anti-Smooth Muscle | Abcam | ab265588 |
| Actin (SMA) antibody | | |
| Anti-SOX2 antibody | Abcam | ab92494 |
| Anti-SOX10 antibody | Abcam | ab212843 |
| [SOX10/991] | | |
| Anti-Synaptophysin | Abcam | ab32127 |
| antibody | | |
| Anti-α Tubulin antibody | Cell Signaling | 3873S |
| | Technologies | |
| Anti-β III Tubulin | Abcam | ab78078 |
| antibody [2G10] | | |
| Alexa Fluor 488 goat | Fisher Scientific | A11029 |
| anti-mouse antibody | | |
| Alexa Fluor 568 goat | Fisher Scientific | A11011 |
| anti-rabbit antibody | | |
| Alexa Fluor 647 goat | Abcam | ab150115 |
| anti-mouse antibody | | |
| Biotinylated anti mouse | Fisher Scientific | BA9200 |
| secondary antibody | | |
| Biotinylated anti rabbit | Vector Laboratories | BA-1000 |
| secondary antibody | | |
| Senescence β- | Cell Signaling | 9860S |
| Galactosidase staining kit | Technologies | |
| Stem Light ™ | Cell Signaling | 9656S |
| Pluripotency antibody kit | Technologies | |
| [OCT-4A (C30A3), | | |
| SOX2 (D6D9), NANOG | | |
| (D73G4) XP, SSEA | | |
| (MC813), TRA-1-60, | | |
| TRA-1-81)] | | |

Immunostaining was performed using the primary antibodies listed in TABLE 1 with appropriate Alexa Fluor-conjugated secondary antibodies. Bis-benzamide (Hoechst) was used as a nuclear counterstain.

Immunocytochemistry

Immunocytochemistry was performed on hiPSCs, iNPCs, iGRPs, iOPCs and astrocytes using the primary antibodies described in Additional file 1: Table 51. Briefly, adherent cells were washed, fixed with 4% paraformaldehyde, permeabilized with 0.5% Triton X-100 in PBS, before blocking in 10% goat serum and incubation overnight at 4° C. in primary antibodies diluted in 2% goat serum at the manufacturer's suggested concentrations. Appropriate secondary Alexa-Fluor-conjugated secondary antibodies diluted to 1:200 were employed and bis-benzamide (Hoechst) was used as a nuclear counterstain. Cells were mounted with Immu-mount (Fisher) and imaged on a Leica DMi1 fluorescent microscope using the Leica LAS X software, as per manufacturer's instructions.

BrdU Proliferation, RAS Activity, cAMP, and Cxcl10 ELISA

RAS activity (ThermoFisher), cAMP (Fisher) detection, BrdU proliferation assays (Roche) and direct cell counting were performed according to the manufacturer's instructions. Cxcl10 (Ray Biotech) ELISA assays were performed according to the manufacturer's instructions. Each assay was performed using a minimum of three independently generated biological replicates.

Lentivirus Production, Cell Infection and CXCL10 Peptide Treatment

Cxcl10 cDNA (Sino Biological) and lentiviral packaging vectors, or KIAA1549:BRAF adenoviral DNA, were transfected in HEK293T cells using Fugene HD (Promega) following the manufacturer's instructions. Viral supernatants were collected 48 h post-transfection, filtered, and used to directly infect iNPCs and iGRPs for 24 h. Cxcl10-GFP expression was confirmed by Western blotting as previously described. KIAA1549:BRAF-infected cells were puromycin-selected for 2 weeks prior to further expansion. Infected cells were used for intracranial injections in Rag1^{-/-} neonatal mice. For cell proliferation, cell death and differentiation analyses, iNPCs were treated with 25 or 100 pg/mL of murine recombinant Cxcl10 (PeproTech) for 24 h. Each experiment was performed a minimum of three times on independently generated samples.

RNA Extraction, Quantitative Real-Time PCR and RNA Sequencing And Analysis

RNA was extracted using a QIAGEN RNeasy mini-kit from snap-frozen brainstem tissues of 1-month-old adult mice following the manufacturer's instructions (QIAGEN). For quantitative real-time PCR (qPCR) studies, total RNA was reverse-transcribed using the High-Capacity cDNA Reverse Transcription Kit protocol following the manufacturer's instructions (Thermo Fisher Scientific). qPCR was performed on a Bio-Rad CFX thermocycler using pre-designed TaqMan Gene Expression Assays (Cxcl10, Chil3, CD59a, Gfap; see e.g., TABLE 2) and a commercially available Taqman mastermix (Thermo Fisher Scientific) following the manufacturer's instructions.

TABLE 2
Primers used.

| Primer | Manufacturer | Catalog Number |
|---|---|---|
| Mouse CD59a - TaqMan ® Gene | ThermoFisher | Mm00483149_m1 |
| Expression Assay FAM-MGB | Scientific | |
| Mouse Chil3 - TaqMan ® Gene | ThermoFisher | Mm00657889_mH |
| Expression Assay FAM-MGB | Scientific | |
| Mouse Cxcl10 - TaqMan ® Gene | ThermoFisher | Mm00445235_m1 |
| Expression Assay FAM-MGB | Scientific | |
| Mouse Gapdh - TaqMan ® Gene | ThermoFisher | Mm99999915_g1 |
| Expression Assay FAM-MGB | Scientific | |

Relative transcript expression was calculated using the ΔΔCT analysis method and normalized to Gapdh as an internal control following the manufacturer's instructions (ThermoFisher). For RNA sequencing, total RNA from three C57BL/6J and three Rag1^{-/-} brainstem samples was submitted to Washington University Genome Technology Access Center (GTAC). Samples were prepared according to the library kit manufacturer's protocol, indexed, pooled, and sequenced on an Illumina HiSeq. Base calls and demultiplexing were performed with Illumina bcl2fastq software and a custom python demultiplexing program with a maximum of one mismatch in the indexing read. The analysis was generated using Partek Flow software, version 8.0. RNA sequencing reads were aligned to the mm10-RefSeq Transcripts 83 assembly with STAR version 2.5.3a. Gene counts and isoform expression were derived from the annotation model output. Sequencing performance was assessed for the total number of aligned reads, total number of uniquely aligned reads, and features detected. Normalization size factors were calculated for all gene counts by CPM to adjust for differences in library size. Gene specific analysis was then performed using the lognormal with shrinkage model (limma-trend method) to analyze for differences between Rag1^{-/-} and control C57 mice. The results were filtered for only those genes with p values≤0.05 and log foldchanges≥±2. Principal component analysis was conducted in Partek Flow using normalized gene counts. RNA sequencing data have been deposited in the GEO portal (GSE174624).

T Cell Isolation and Culture

4-6-week-old C57BL/6J and Rag1^{-/-} mouse spleens were homogenized into single cell suspensions by digestion in PBS containing 0.1% BSA and 0.6% sodium citrate. The homogenates were subsequently washed and incubated with 120 Kunitz units of DNase I for 15 min following red blood cell lysis (eBioscience). Cells were then filtered through a 30 μM cell strainer to obtain a single cell suspension. CD4+ and CD8+ T cells were isolated using CD8a (Miltenyi Biotech) or CD4 (Miltenyi Biotech) T cell isolation kits, respectively. T cells were maintained at 2.5×10⁶ cells ml⁻¹ in RPM1-1640 medium supplemented with 10% FBS and 1% penicillin/ streptomycin. T cells were activated by 1.25 μg ml⁻¹ anti-mouse CD3 (Fisher Scientific) and 2 μg ml⁻¹ anti-mouse CD28 (Fisher Scientific) antibody treatment for 48 h. T cell conditioned media (TCM) was collected both from nonactivated and activated T cells following 22 μM filtration for subsequent chemokine assay, ELISA, and co-culture experiments.

Astrocyte Isolation and Culture

4- to 6-week-old C57BL/6 J and Rag1^{-/-} mice were transcardially perfused with DPBS and whole brains collected following the removal of the cerebellum and olfactory bulbs. A single cell suspension was generated using the Miltenyi Biotech adult brain dissociation kit following the manufacturer's instructions. The resulting cells were seeded in Poly-l-Lysine-coated (Millipore Sigma) T75 flasks and incubated in Minimal Essential Medium supplemented with 1 mM 1-glutamine, 1 mM sodium pyruvate, 0.6% D-(+)-glucose, 100 μg ml⁻¹ penicillin/ streptomycin and 10% FBS. A complete media change was performed after 24 h and every third day after that for 12-14 days, in order to obtain mixed glial cultures composed of microglia on an astrocyte monolayer. To release the microglia, flasks were mechanically shaken at 200 rpm for 5 h at 37° C., and the microglia-containing supernatant discarded. Astrocyte-enriched cultures containing >95% GFAP-positive astrocyte monolayers were passaged with 0.1% trypsin (Invitrogen) for subsequent experiments. Conditioned media from naive or TCM-treated astrocytes was collected following 22 μM filtration.

Quantification and Statistical Analysis

All statistical tests were performed using GraphPad Prism 5 software. 2-tailed Student's t-tests, or one-way analysis of variance (ANOVA) with Bonferroni post-test correction using GraphPad Prism 5 software. Statistical significance was set at p<0.05, and individual p values are indicated within each graphical figure. A minimum of 3 independently generated biological replicates was employed for each of the analyses. Numbers (n) are noted for each individual analysis.

What is claimed is:

1. A humanized xenograft animal model comprising an animal engineered to be deficient in Cxcl10 and a population of human cells in the nervous system of the animal.

2. The humanized xenograft animal model of claim 1, wherein the animal engineered to be deficient in Cxcl10 has a homozygous mutation in Cxcl10.

3. The humanized xenograft animal model of claim 1, wherein the animal engineered to be deficient in Cxcl10 has a homozygous mutation in Rag1.

4. The humanized xenograft animal model of claim 1, wherein the population of human cells comprises:
patient-derived low-grade glioma (LGG) cells; or
cells comprising a mutation in the NF1 gene or expressing KIAA1549:BRAF or cells derived therefrom.

5. The humanized xenograft animal model of claim 4, wherein the patient-derived LGG cells are derived from a human subject having at least one of a LGG, sporadic LGG, NF1 tumor predisposition syndrome, NF1-associated optic pathway glioma (NF1-OPG), grade 1 pilocytic astrocytoma (PA), or BRAF-driven sporadic LGG.

6. The humanized xenograft animal model of claim 5, wherein the human subject is a pediatric human subject.

7. The humanized xenograft animal model of claim 4, wherein the population of human cells comprises human induced pluripotent stem cells (hiPSCs).

8. The humanized xenograft animal model of claim 4, wherein the population of human cells comprises (hiPSC)-derived neural progenitor cells.

9. The humanized xenograft animal model of claim 4, wherein the population of human cells comprises hiPSC-derived glial restricted progenitors (iGRPs), or hiPSC-derived oligodendrocyte progenitors (iOPCs).

10. The humanized xenograft animal model of claim 4, wherein the mutation in the NF1 gene is c.2041C>T or c.6513T>A.

11. The humanized xenograft animal model of claim 1, wherein the population of human cells are located in a LGG xenograft or glioma-like lesion in a brain of the animal.

12. The humanized xenograft animal model of claim 11, wherein the LGG xenograft or glioma-like lesion is hypercellular, parenchymal with exophytic components, anterior or lateral to midbrain or brainstem tissue or anterior to the cerebellum, well-circumscribed, or contains GFAP- and OLIG2-immunopositive cells.

13. The humanized xenograft animal model of claim 1, wherein the animal model is a mouse model or a rat model.

14. A method for screening an anticancer agent in a xenograft animal model, the method comprising:
administering an anticancer agent candidate to the xenograft animal model of claim 1; and
analyzing growth or metastasis of a cancer in the xenograft animal model to determine therapeutic efficacy of the anticancer agent candidate.

15. A method of growing a humanized low-grade glioma (LGG) xenograft in a host animal comprising:
providing a host animal deficient in Cxcl10, and
administering an amount of humanized LGG cells to the host animal sufficient to grow a LGG xenograft or glioma-like lesion in the host animal.

16. The method of claim 15, wherein the host animal has a homozygous mutation in Cxcl10 or Rag1.

17. The method of claim 15, wherein the host animal is deficient in CD4+ T cells.

18. The method of claim 15, wherein the LGG cells are derived from a human subject having an LGG or an isolated population of cells comprising a mutation in the NF1 gene or expressing a KIAA1549:BRAF fusion gene.

19. The method of claim 18, wherein the isolated population of cells comprises hiPSCs.

20. The method of claim 18, wherein the isolated population of cells comprises (hiPSC)-derived neural progenitor cells.

21. The method of claim 18, wherein the isolated population of cells comprises hiPSC-derived glial restricted progenitors (iGRPs) or hiPSC-derived oligodendrocyte progenitors (iOPCs).

22. The method of claim 18, wherein the mutation in the NF1 gene is c.2041C>T or c.6513T>A.

23. The method of claim 18, wherein the human subject has at least one of a sporadic LGG, NF1 tumor predisposition syndrome, NF1-associated optic pathway glioma (NF1-OPG), grade 1 pilocytic astrocytoma (PA), or BRAF-driven sporadic LGG.

24. The method of claim 18, wherein the human subject is a pediatric human subject.

25. The method of claim 15, wherein the host animal is a mouse model or a rat model.

26. The method of claim 15, wherein the LGG cells are administered by intracranial injection.

27. The method of claim 15, wherein the LGG xenograft or glioma-like lesion is hypercellular, parenchymal with exophytic components, anterior or lateral to midbrain or brainstem tissue or anterior to the cerebellum, well-circumscribed, or contains GFAP- and OLIG2-immunopositive cells.

28. A method of engineering cells comprising:
obtaining human induced pluripotent stem cells (hiPSCs); and
introducing an Nf1 mutation into the hiPSCs; or
introducing a KIAA1549:BRAF fusion gene into the hiPSCs.

29. The method of claim 28, wherein the Nf1 mutation is c.2041C>T or c.6513T>A.

30. The method of claim 28, further comprising differentiating the hiPSCs into neural progenitor cells (iNPCs).

31. The method of claim 30, further comprising differentiating the iNPCs into glial restricted progenitors (iGRPs) or oligodendrocyte progenitor cells (iOPCs).
